(19) United States
(12) Patent Application Publication
(10) Pub. No.: US 2006/0241074 A1
(43) Pub. Date: Oct. 26, 2006

(54) Methods for treatment of pain

(75) Inventors: Clifford Woolf, Newton, MA (US); Michael Costigan, Somerville, MA (US); Robert Griffin, Charlestown, MA (US)

Correspondence Address: PALMER & DODGE, LLP;KATHLEEN M. WILLIAMS, 111 HUNTINGTON AVENUE, BOSTON, MA, 02199, US

(73) Assignee: The General Hospital Corporation

(21) Appl. No.: 11/231,114

(22) Filed: Sep. 20, 2005

Related U.S. Application Data
(63) Continuation-in-part of application No. 10/219,051, filed on Aug. 14, 2002.
(63) Continuation-in-part of application No. PCT/US04/42360, filed on Dec. 17, 2004.
(60) Provisional application No. 60/312,147, filed on Aug. 14, 2001.
(60) Provisional application No. 60/346,382, filed on Nov. 1, 2001.
(60) Provisional application No. 60/333,347, filed on Nov. 26, 2001.
(60) Provisional application No. 60/531,341, filed on Dec. 19, 2003.

(51) Int. Cl. A61K 48/00; A61K 38/17; A61K 31/727

(52) U.S. Cl. 514/44; 514/12; 514/56

(57) ABSTRACT

The present invention relates to a method for the treatment or prevention of pain by administering to an animal an agent that decreases the activity of the complement cascade.

### METHODS FOR TREATMENT OF PAIN

This application claims priority as a Continuation in Part of U.S. Ser. No. 10/219,051, filed Aug. 14, 2002, which claims priority to U.S. Ser. No. 60/312,147, filed Aug. 14, 2001; U.S. Ser. No. 60/346,382, filed Nov. 1, 2001; and U.S. Ser. No. 60/333,347, filed Nov. 26, 2001 and as a Continuation in Part of International application number PCT/US04/042360, filed Dec. 14, 2004, which claims priority to U.S. Ser. No. 60/531,341, filed Dec. 19, 2003. The contents of each of the foregoing are incorporated herein in their entirety.

### BACKGROUND

Pain is a state-dependent sensory experience which can be represented by a constellation of distinct types of pain including, neuropathic pain, inflammatory pain, dysfunctional pain and nociceptive pain. Current therapy is, however, either relatively ineffective or accompanied by substantial side effects (Sindrup and Jensen, 1999 Pain 83: 389). Most of the primary forms of pain therapy have been discovered either empirically through folk medicine, or serendipitously. These forms of treatment include opiates, non-steroidal anti-inflammatory drugs (NSAIDS), local anesthetics, anticonvulsants, and tricyclic antidepressants (TCAs).

Recently there has been a great deal of progress in understanding the mechanisms that produce pain (McCleskey and Gold, 1999, Annu. Rev. Physiol. 61: 835; Woolf and Salter, 2000, Science 288: 1765; Mogil et al., 2000, Annu. Rev. Neurosci. 23: 777). It is increasingly clear that multiple mechanisms operating at different sites, and with different temporal profiles, are involved and, thus, a strategy that attempts to identify and treat the mechanisms present in a given patient would be advantageous (Woolf and Mannion, 1999, Lancet 353: 1959; Woolf and Decosterd, 1999, Pain 82: 1). It would be greatly useful to develop a method which permits regulation of pain at its mechanistic source, and which provides an effective treatment for pain, particularly neuropathic pain.

### SUMMARY OF THE INVENTION

The invention is based, in part, on the observation that specific elements of the complement cascade are significantly upregulated across several different models of peripheral neuropathic pain. Without being bound to one particular theory, this observation suggests that the complement pathway may be a key point of manipulation for developing new pain therapies.

The present invention provides, therefore, a method for the treatment of pain in an animal. The invention includes a method of treating pain in an animal by administering to the animal and antisense polynucleotide capable of inhibiting the expression of a polynucleotide sequence that encodes a component of the complement cascade.

The invention also provides for a method of treating pain in an animal by administering a double stranded RNA molecule to the animal wherein one of the strands of the double stranded RNA molecule is identical to at least 10 contiguous residues of an mRNA transcript obtained from a polynucleotide sequence encoding a of the component of the complement cascade. For example, one of the strands of the double stranded RNA molecule can be identical to 10 or more, 20, 30, 40, 50, 60, 70, 80, and up to 90 or more contiguous residues of an mRNA transcript obtained from a polynucleotide sequence encoding a component of the complement cascade.

The invention further provides a method for treating pain in an animal by administering an agent which decreases the activity of the complement cascade, sequesters components of the cascade or blocks their assembly or actions on receptors. An agent, useful in the invention, can decrease the activity of the complement cascade by decreasing the activity or available amounts of a component of the complement cascade. Because the complement system operates as a cascade, decreasing the activity or availability of a particular component of the cascade will decrease the activity of all the components downstream in the cascade. Compounds which could be used as agents that decrease the activity or availability of the complement cascade include, but are not limited to, soluble complement receptor type 1, soluble complement receptor type 1 lacking long homologous repeat-A, soluble complement receptor type 1-sialyl lewis, complement receptor type 2, soluble decay accelerating factor, soluble membrane cofactor protein, soluble CD59, decay accelerating factor-CD59 hybrid, membrane cofactor protein-decay accelerating factor hybrid, C1 inhibitor, C1q receptor, C089, PR226, CBP2, DFP, BCX-1470, TKIXc, K-76 COOH, FUT-175, PS-oligo, Glycyrrhizin, GR-2II, AGIIb-1, AR-2IIa, Rosmarinic acid, BR-5-I, Fucan, complestatin, decorin, dextran, heparin, LU51198, GCRF, CSPG, C4 inactivator, compstatin, CR1 (CD35), CD2 (CD21), MCP (CD46), DAF (CD55), factor H, C3BP, Crry, TP-10, plasma-derived protein C1 esterase inhibitor, vaccinia virus complement control protein, AcF[OPdCHaWR], CGS32359, 3D53, SB-290157, and cobra venom factor.

The invention also provides a method for treating pain in an animal by administering a therapeutically effective amount of an antibody polypeptide that binds to a component of the complement cascade. Antibodies which bind to a component of the complement cascade include, but are not limited to antibody polypeptides that bind to MBL, factor D, C5, C5a, and C8. Based on the level of skill of those in the art, however, antibody polypeptides could be generated which would bind to any of the specific members of the complement cascade.

The invention also provides pharmaceutical formulations which include the antisense polynucleotide, double stranded RNA, antibody polypeptide, and/or compounds described above, and a carrier.

Definitions

As used herein the term “component of the complement cascade” refers to a protein: including an enzyme, or proenzyme that is active in the complement cascade and classically defined as part of the complement cascade. The complement cascade and components of the complement cascade are known in the art, and are described, for example, in Morgan, 1999, Crit. Rev. Immunol. 19:173-198. Components of the complement cascade include, but are not limited to C1q alpha, C1q beta, C1q gamma, C1r, C1s, C1q binding protein, C2, C4, C4a, C4b, Mbl2, Masp1, Masp2, bf, properdin, And, C3, C3a, C3b, C3ar1, C5, C5a, C5b, C5rl, C6, C7, C8b, C8a, C9, C1 inhibitor, C4bpa, C4bp-ps1, Cfh, Cfi, Vtn, Crry, Daf1, mcp, Cd59, S100b.

As used herein the term “nerve injury pain model” includes three alternate nerve injury pain models by which differential expression can be determined according to the invention: spared nerve injury (SNI), spinal segmental nerve lesion, and chronic constriction injury.

As used herein, a “spared nerve injury pain model” (SNI) refers to a situation in which one of the terminal branches of the sciatic nerve is spared from axotomy (Decosterd and Woolf, 2000 Pain 87: 149). The SNI procedure comprises an axotomy and ligation of the tibial and common peronial nerves leaving the sural nerve intact.

As used herein, a “spinal segmental nerve lesion” (also called the “Chung” model) and “chronic constriction injury” (CCI) refer to two types of “neuropathic pain models” useful in the present invention. Both models are well known to those of skill in the art (See, for example Kim and Chung, 1992 Pain 50: 355; and Bennett, 1993 Muscle Nerve 16: 1040 for a description of the “segmental nerve lesion” and “chronic constriction” respectively). A “segmental nerve lesion” and/or “chronic constriction injury” neuropathic pain model may be evaluated for the presence of “pain” using any of the behavioral, electrophysiological, and/or neurochemical criteria described below.

As used herein, an “inflammatory pain model” refers to a situation in which an animal is subjected to pain, as defined herein, by the induction of peripheral tissue inflammation (Stein et al., (1988) Pharmacol Biochem Behav 31: 445-451; Woolf et al., (1994) Neurosci. 62, 327-331). The inflammation can be produced by injection of an irritant such as complete Freunds adjuvant (CFA), carrageenan, turpentine, croton oil, and the like into the skin, subcutaneously, into a muscle, into a joint, or into a visceral organ. In addition, an “inflammatory pain model” can be produced by the administration of cytokines or inflammatory mediators such as lippopolysoccharide (LPS), or nerve growth factor (NGF) which can mimic the effects of inflammation. An “inflammatory pain model” can be evaluated for the presence of “pain” using behavioral, electrophysiological, and/or neurochemical criteria as described below.

As used herein, “nerve tissue” refers to animal tissue comprising nerve cells, the neuropil, glia, neural inflammatory cells, and endothelial cells in contact with “nerve tissue”. “Nerve cells” may be any type of nerve cell known to those of skill in the art including, but not limited to motor neurons, sensory neurons, enteric neurons, sympathetic neurons, parasympathetic neurons, and central nervous system neurons. “Glial cells” useful in the present invention include, but are not limited to astrocytes, Schwan cells, and oligodendrocytes. “Neural inflammatory cells” useful in the present invention include, but are not limited to cells of myeloid origin including macrophages and microglia. Preferably, “nerve tissue” as used herein refers to nerve cells obtained from the dorsal root ganglion, or dorsal horn of the spinal cord.

As used herein, “sensory neuron” refers to any sensory neuron in an animal. A “sensory neuron” can be a peripheral sensory neuron, central sensory neuron, or enteric sensory neuron. A “sensory neuron” includes all parts of a neuron including, but not limited to the cell body, axon, and dendrite(s). A “sensory neuron” refers to a neuron which receives and transmits information (encoded by a combination of action potentials, neurotransmitters and neuropeptides) relating to sensory input, including, but not limited tonoxious stimuli, heat, touch, cold, pressure, vibration, etc. Examples of “sensory neurons” include, but are not limited to dorsal root ganglion neurons, dorsal horn neurons of the spinal cord, autonomic neurons, trigeminal ganglion neurons, and the like.

As used herein, “animal” refers to a organism classified within the phylogenetic kingdom Animalia. As used herein, an “animal” preferably refers to a mammal. Animals, useful in the present invention, include, but are not limited to mammals, marsupials, mice, dogs, cats, cows, humans, deer, horses, sheep, livestock, and the like.

As used herein, “polynucleotide” refers to a polymeric form of nucleotides of 2 up to 1,000 bases in length, or even more, either ribonucleotides or deoxyribonucleotides or a modified form of either type of nucleotide. The term includes single and double stranded forms of DNA. The term is synonymous with “oligonucleotide”.

As used herein, “polypeptide” refers to any kind of polypeptide such as peptides, human proteins, fragments of human proteins, proteins or fragments of proteins from non-human sources, engineered versions proteins or fragments of proteins, enzymes, antigens, drugs, molecules involved in cell signaling, such as receptor molecules, antibodies, including polypeptides of the immunoglobulin superfamily, such as antibody polypeptides or T-cell receptor polypeptides.

As used herein, “inhibits the expression” of a polynucleotide sequence refers to inhibiting or blocking the transcription of a gene in response to a treatment by at least 10% compared to the amount of gene expression in the absence of said treatment. “Inhibits the expression” refers to inhibiting or blocking transcription of a gene by at least 10% or more, 20% or more, 30%, 40%, 50%, 60%, 70%, 80%, 90% or more, and up to 100%, or complete inhibition of transcription. The “level of expression” may be measured by hybridization analysis using labeled target nucleic acids according to methods well known in the art (see, for example, Ausubel et al., Short Protocols in Molecular Biology, 3^{rd} Ed. 1995, John Wiley and Sons, Inc.). The label on the target nucleic acid is a luminescent label, an enzymatic label, a radioactive label, a chemical label or a physical label. Preferably, the target nucleic acids are labeled with a fluorescent molecule. Preferred fluorescent labels include fluorescein, amino coumarin acetic acid, tetramethylrhodamine isothiocyanate (TRITC), Texas Red, Cy3 and Cy5. Alternatively, the level of expression of a polynucleotide sequence of the invention may be measured by other suitable methods such as PCR, quantitative PCR, Northern Analysis, Southern Analysis and other methods which are known to those of skill in the art.

As used herein, the term “therapeutically effective amount” refers to that amount of a compound, antibody, antisense polynucleotide, or double stranded RNA molecule that is required to reduce the pain or the symptoms thereof in an animal, for example, at least by 10%, 20%, 30%, 40%, 50%, 60%, 70%, 80%, 90%, and up to 100% or more, compared to an animal not treated with the same compound, antibody, antisense polynucleotide, or double stranded RNA molecule, or compared to the same animal before the treatment with the compound, antibody, antisense polynucleotide, or double stranded RNA molecule. The term “therapeutically effective amount” also refers to an amount of a compound, antibody, antisense polynucleotide, or double stranded RNA molecule, that enhances or improves the prophylactic or therapeutic effect(s) of another therapy by at least 10% or more, 20% or more, 305, 40%, 50%, 60%, 70%, 80%, 90%, and up to 100% or more. Accordingly, pain is “treated” when the level of pain is decreased, as measured using any of the pain assays described herein, and/or any clinically relevant scoring method known to those of skill in the art, by at least 10% or more, 20%, 30%, 40%, 50%, 60%, 70%, 80%, 90%, and up to 100% or more relative to the level of pain in an animal not treated with an agent according to the invention. As used herein, pain is “prevented” where the onset or perception of pain in response to a stimulus (e.g, a stimulus utilized in a pain model described herein, or where the stimulus is, for example, an injury, surgery, or other physical insult that generally results in the perception of pain by an individual) either does not occur in an animal that has been administered an agent that decreases the activity of the complement cascade, or where the time between the stimulus and the onset or perception of pain is increased relative to an animal that has not been treated with an agent the decreases the activity of the complement cascade.

As used herein, the term “decrease the activity of the complement cascade” refers to a decrease in the activity of the cascade of at least 10% or more, including 20%, 30%, 40%, 50%, 60%, 70%, 80%, 90%, and up to 100% or more, in response to an agent relative to the activity of the cascade in the absence of the agent. As used herein, the “activity of the complement cascade” refers to the activity of the individual components of the cascade (for example, the activity of C3b is to form part of the C5 convertase, and to bind to cells making them more susceptible to phagocytosis), and also refers to the activity of the fmal effector molecules of the cascade (e.g., activity of the C5b6789 membrane attack complex to cause osmotic lysis of a cell). The activity of the complement cascade, and of the individual components of the complement cascade is known in the art, and may be found, for example, in Makrides, S. C. (1998, Pharmacological Reviews 50:59-78) and Janeway et al. (1999, Immunobiology, Garland Publishing NY, N.Y.). The activities of the components of the complement cascade can be measured using assays which are well known in the art and described in more detail below. The activity of a component of the complement cascade also refers to the availability or assembly of the component of the cascade. As used herein, an “agent that decreases the activity of the complement cascade: refers to a protein, antibody, enzyme, small molecule, antisense RNA, or siRNA that decreases the activity or available amount of a component of the complement cascade, and/or decreases the activity or blocks assembly of the ultimate effector molecules or their binding to receptors of the complement cascade by at least 10% or more, including 20%, 30%, 40%, 50%, 60%, 70%, 80%, 90%, and up to 100% or more relative to the activity of the complement cascade in the absence of the agent.

As used herein, the term “antibody polypeptide” refers to a polypeptide which either is an antibody or is a part of an antibody, modified or unmodified, which retains the ability to specifically bind antigen. Thus, the term antibody polypeptide includes a whole antibody, an antigen-binding heavy chain, light chain, heavy chain-light chain dimer, Fab fragment, F(ab′)2 fragment, dAb, or an Fv fragment, including a single chain Fv (scFv). The phrase “antibody polypeptide” is intended to encompass recombinant fusion polypeptides that comprise an antibody polypeptide sequence that retains the ability to specifically bind antigen in the context of the fusion.

As used herein, the term “specifically binds” refers to the interaction of two molecules, e.g., an antibody polypeptide and a protein or peptide, wherein the interaction is dependent upon the presence of particular structures on the respective molecules. For example, when the two molecules are protein molecules, a structure on the first molecule recognizes and binds to a structure on the second molecule, rather than to proteins in general. “Specific binding”, as the term is used herein, means that a molecule binds its specific binding partner with at least 2-fold greater affinity, and preferably at least 10-fold, 20-fold, 50-fold, 100-fold or higher affinity than it binds a non-specific molecule. Alternatively, “specifically binds” as used herein refers to the binding of two protein molecules to each other with a dissociation constant (K_{d}) of 1 μM or lower. For example, the affinity or K_{d} for a specific binding interaction can be about 1 μM, or lower, about 500 nM or lower, and about 300 nM or lower. Preferably the K_{d} for a specific binding interaction is about 300 nM or lower. Specific binding between two molecules (e.g., protein molecules) can be measured using methods known in the art. For example, specific binding may be determined as measured by surface plasmon resonance analysis using, for example, a BIAcore™ surface plasmon resonance system and BIAcore™ kinetic evaluation software (e.g., version 2.1).

The invention is based, in part, on the discovery that certain components of the complement cascade are differentially expressed in animals subjected to pain. A nucleic acid molecule of the present invention is differentially expressed if it demonstrates at least a 1.4 fold change in expression levels across three replicate assays in an animal subjected to the neuropathic or inflammation pain as described herein relative to an animal not subjected to the same pain. Preferably, “differential expression” is measured in either a nerve injury model, or inflammation pain model, or both, at multiple time points after an animal has been subjected to pain. “Differential expression” is further measured in at least three replicate samples for each time point, and for multiple pain models (e.g. nerve injury models, an inflammation models), such that a statistical evaluation may be made of the significance of the differential expression. Accordingly, a polynucleotide sequence is “differentially expressed” as determined by microarray hybridization if the mean intensity level of the signal on the array is greater than 1000 for at least one data point, and if it is differentially expressed by at least 1.4 fold across triplicate assays with a P-value of less than 0.05 in at least two independent results in any of the pain models versus the corresponding control.

As used herein a polynucleotide sequence is “differentially expressed” if it is over or under expressed by at least 1.4 fold over at least three replicate assays with a statistical significance of P<0.05, in at least two of the pain models tested. In a further embodiment, a polynucleotide sequence is “differentially expressed” if it is over or under expressed by at least 1.4 fold over at least three replicate assays with a statistical significance of P<0.05 in at least two of the pain models tested, and the sequence is reasonably determined by one of skill in the art, based on its known or deduced function, to have a role in the production of pain by changing the membrane properties (e.g., membrane potential, capacitance, membrane resistance, etc.), excitability, survival, chemical composition and/or structure connectivity of neurons in pain circuits. In a still further embodiment, a polynucleotide sequence is “differentially expressed” if it is over or under expressed by at least 1.4 fold over at least three replicate assays with a statistical significance of P<0.05 in only one of the pain models tested, but the sequence is reasonably determined by one of skill in the art, based on its known or deduced function, to have a substantial role in the production of pain by changing the membrane properties (e.g., membrane potential, capacitance, membrane resistance, etc.), excitability, survival, chemical composition and/or structure connectivity of neurons in pain circuits.

### BRIEF DESCRIPTION OF THE DRAWINGS

FIG. 1(A) shows a dendrogram display of hierarchical cluster analysis of the microarray data set. Stem length is proportional to the dissimilarity between arrays or clusters of arrays. FIG. 1(B) shows a multidimensional scaling display of the dissimilarities among the microarrays. FIG. 1(C) shows a venn diagram showing the number of genes meeting fold difference and statistical thresholds according to tissue (DRG or DH) and model (SNI, CCI, or SNL).

FIG. 2 shows temporal expression patterns of the genes regulated after SNI, CCI, or SNL. Each gene was normalized according to mean 0, standard deviation 1, then subjected to k-means clustering. Expression intensity is shown where increasing greyscale intensity indicates increasing relative expression level. Inset plots show two examples of clusters. (A) DRG. (B) DH.

FIG. 3 shows genes meeting criteria for differential expression in SNI, CCI, and SNL models, in either the DRG or the DH, limited to the genes involved in immune system function.

FIG. 4 shows an in situ hybridization showing expression of complement genes C1qb, C4, and C3 in naïve spinal cord, and spinal cord tissue three, seven, and forty days after SNI injury. The inset number is the fold difference calculated using the microarray.

FIG. 5 shows fluorescent in situ hybridization for C3, C4, or C1q was carried out in the spinal cord dorsal horn. The in situ signal colocalizes with immunofluorescent staining of IBA1, a microglialmarker. The in situ signals did not colocalize with either NeuN (a neuronal marker) or GFAP (a marker of astrocytes). Staining of C3 and C4 was done at 7d post-injury, while staining of C1q was done at 3d post-injury.

FIG. 6(A) shows a photomontage showing increased IBA1 immunofluorescence (blue) ipsilateral to SNI injury, 5d post-injury. Fluorescent staining for isolectin B4 binding (a marker of sensory fiber terminals) is also shown (green). FIG. 6(B) shows complement C3 immunoreactivity (red) is increased in the dorsal horn ipsilateral to SNI, shown at 5d post-injury. Both cellular staining and diffuse interstitial staining are present. FIG. 6(C) shows cellular C3 staining (red) colocalizes with IBA (blue). Arrows (white) indicate double-positive cells.

FIG. 7 shows results from experiments in which rats with the SNI injury were treated with intrathecal cobra venom factor. Osmotic pumps were placed 24 hrs prior to the injury. FIG. 7(A) shows the Von Frey mechanical threshold response. FIG. 7(B) shows the pinprick response. Complement C5 deficient mice were subjected to SNI. FIG. 7(C) shows a Von Frey response. FIG. 7(D) shows a pinprick response. For A-D, data is shown as mean ±SEM, with the black line corresponding to vehicle (0.9% NaCl) only, and the red line corresponding to CVF treatment. FIG. 7(E) shows in situ hybridization for CD59 in spinal cord. FIG. 7(F) shows in situ hybridization for CD59 in DRG.

FIG. 8 shows a summary of the data. Red boxes indicate complement components demonstrated by in situ hybridization or immunohistochemistry. Green boxes indicate complement components with action supported by behavioral testing of CVF treated rats or C5 deficient mice. Black lines indicate complement cascade connections. Blue lines indicate hypothesized relationship between peripheral nerve injury, complement activation, and pain.

### DETAILED DESCRIPTION OF THE INVENTION

The present invention is based, in part, on the discovery that certain genes that encode components of the complement cascade are significantly upregulated in several animal models of pain. Specifically, the complement component genes are upregulated in models of neuropathic pain, including the spared nerve injury, chronic constriction injury, and spinal nerve ligation models of peripheral neuropathic pain. The upregulation of these complement component genes was first reported in co-pending applications U.S. Ser. No. 10/219,051, filed Aug. 14, 2002 (which claims priority to U.S. Ser. Nos. 60/312,147; 60/346,382; and 60/333,347), and International Application PCT/US04/042360, filed Dec. 14, 2004 (which claims priority to U.S. Ser. No. 60/531,341), the contents of which are incorporated herein in their entirety. More particularly, the complement genes for C1q, C3 and C4 (identified as gene ID numbers C1q: X71127; C4: U42719 (both disclosed in U.S. Ser. No. 10/219,051); and C3: M29866 (disclosed in PCT/US04/042360) were shown to be significantly upregulated in several animal models of pain (i.e., by at least 1.4 fold, with a statistical significance of at least p<0.05). The present invention is based also, in part, on the discovery that inhibition of the complement cascade (e.g., by blocking the activity of C3 or C5) significantly attenuated peripheral neuropathic pain. Accordingly, the invention provides a method for the treatment of pain by administering to an animal a therapeutically effective amount of an agent which decreases the activity of the complement cascade. Agents useful for the inhibition of the complement cascade are known in the art and are described in more detail below.

Pain

The present invention includes polynucleotides which are differentially expressed in (a) an animal that is subjected to pain relative to (b) an animal not subjected to pain. According to the invention, the pain to which the animals of (a) and (b) are subjected is the same pain, that is, if a polynucleotide is differentially expressed in an SNI pain model then the differential expression is relative to the expression of the polynucleotide in an animal which is not an SNI pain model. The present invention also includes methods for the treatment of pain, that is, for example, a decrease in the perception of pain in an animal by decreasing the activity of the complement cascade.

As used herein, “pain” refers to a state-dependent sensory experience generated by the activation of high threshold peripheral sensory neurons, the nociceptors. As used herein, “pain” refers to several different types of pain, including nociceptive or protective pain, inflammatory pain that occurs after tissue damage, and neuropathic pain which occurs after damage to the nervous system. Physiological pain is initiated by sensory nociceptor fibers innervating the peripheral tissues and activated only by noxious stimuli, and is characterized by a high threshold to mechanical and thermal stimuli and rapid, transient responses to such stimuli. Inflammatory and neuropathic pain are characterized by displays of behavior indicating either spontaneous pain, measured by spontaneous flexion, vocalization, biting, or even self mutilation, or abnormal hypersensitivity to normally innocuous stimuli or to noxious stimuli, such as mechanical or thermal stimuli. Regardless of the type of pain, as used herein “pain” can be measured using behavioral criteria, such as thermal and mechanical sensitivity, weight bearing, visceral hypersensitivity, or spontaneous locomotor activity, electrophysiological criteria, such as in vivo or in vitro recordings from primary sensory neurons and central neurons to assess changes in receptive field properties, excitability or synaptic input, or neurochemical criteria, such as changes in the expression or distribution of neurotransmitters, neuropeptides and proteins in primary sensory and central neurons, activation of signal transduction cascades, expression of transcription factors, or phosphorylation of proteins.

Behavioral criteria used to measure “pain” include, but are not limited to mechanical allodynia and hyperalgesia, and temperature allodynia and hyperalgesia. Mechanical allodynia is generally measured using a series of ascending force von Frey monofilaments. The filaments are each assigned a force which must be applied longitudinally across the filament to produce a bend, or bow in the filament. Thus the applied force which causes an animal to withdraw a limb can be measured (Tal and Bennett, 1994 Pain 57: 375). An animal can be said to be experiencing “pain” if the animal demonstrates a withdrawal reflex in response to a force that is reduced by at least 30% compared to the force that elicits a withdrawal reflex in an animal which is not in “pain”. In one embodiment, an animal is said to be experiencing “pain” if the withdrawal reflex in response to a force that is reduced 40%, 50%, 60%, 70%, 80%, 90% and as much as 99% compared to the force required to elicit a similar reflex in a naïve animal.

Mechanical hypersensitivity can be measured by applying a sharp object, such as a pin, to the skin of an animal with a force sufficient to indent, but not penetrate the skin. The duration of withdrawal from the sharp stimulus may then be measured, wherein an increase in the duration of withdrawal is indicative of “pain” (Decostard et al., 1998 Pain 76: 159). For example, an animal can be said to be experiencing “pain” if the withdrawal duration following a sharp stimulus is increased by at least 2 fold compared with an animal that is not experiencing “pain”. In one embodiment, an animal is said to be experiencing “pain” if the withdrawal duration is increased by 3, 4, 5, 6, 7, 8, 9, and up to 10 fold compared to an animal not experiencing “pain”.

Temperature allodynia can be measured by placing a drop of acetone onto the skin surface of an animal using an instrument such as a blunt needle attached to a syringe without touching the skin with the needle. The rapid evaporation of the acetone cools the skin to which it is applied. The duration of the withdrawal response to the cold sensation can then be measured (Choi et al., 1994 Pain 59: 369). An animal can be said to be in “pain” if the withdrawal duration following acetone application is increased by at least 2 fold as compared to an animal that is not experiencing “pain”. According to the invention an animal can be said to be in “pain” if the withdrawal duration following thermal stimulation is increased by 4, 6, 8, 10, 12, 14, 16, 18, and up to 20 fold compared to an animal not experiencing “pain”.

Temperature hyperalgesia can be measured by exposing a portion of the skin surface of an animal, such as the plantar surface of the foot, to a beam of radiant heat through a transparent perspex surface (Hargreaves et al., 1988 Pain 32:77). The duration of withdrawal from the heat stimulus may be measured, wherein an increase in the duration of withdrawal is indicative of “pain”. An animal can be said to be experiencing “pain” if the duration of the withdrawal from the heat stimulus increases by at least 2 fold compared with an animal that is not experiencing “pain”. In addition, an animal can be said to be experiencing “pain” if the duration of the withdrawal from heat stimulus is increased by 3, 4, 5, 6, 7, 8, 9, and up to 10 fold compared with an animal that is not experiencing “pain”.

In addition to the behavioral criteria described above, an animal can be deemed to be experiencing “pain” by measuring electrophysiological changes, in vitro or in vivo, in primary sensory, or central sensory neurons. Electrophysiological changes can include increased neuronal excitability, changes in receptive field input, or increased synaptic input. The technique of measuring cellular physiology is well known to those of skill in the art (see, for example, Hille, 1992 Ion channels of excitable membranes. Sinauer Associates, Inc., Sunderland, Mass.). An increase in neuronal excitability may be identified, for example, by measuring an increase in the number of action potentials per unit time in a given neuron. An animal is said to be experiencing “pain” if there is at least a 2 fold increase in the action potential firing rate compared with an animal that is not experiencing “pain.” In addition, and animal can be said to be experiencing “pain” if the action potential firing rate is increased by, 3, 4, 5, 6, 7, 8, and up to 10 fold compared to an animal that is not experiencing “pain”. An increase in synaptic input to a sensory neuron, either peripheral or central, may be identified, for example, by measuring the rate of excitatory post synaptic potentials (EPSPs) recorded from the neuron. An animal is said to be experiencing “pain” if there is at least a 2 fold, 3, 4, 5, 6, 7, 8, and up to 10 fold increase in the rate of EPSPs recorded from a given neuron compared to an animal that is not experiencing pain.

Alternatively, neurochemical criteria may be used to determine whether or not an animal is experiencing “pain”. For example, an animal which has experienced “pain” will display changes in the expression or distribution of neurotransmitters, neuropeptides and protein in primary sensory and central neurons, activation of signal transduction cascades, expression of transcription factors, or phosphorylation of proteins. Gene and protein expression, and phosphorylation of proteins such as transcription factors may be measured using a number of techniques known to those of skill in the art including but not limited to PCR, Southern analysis, Northern analysis, Western analysis, immunohistochemistry, and the like. Examples of signal transduction pathway constituents which may be activated in an animal which is experiencing pain include, but are not limited to ERK, p38, and CREB. Examples of genes which may exhibit enhanced expression include immediate early genes such as c-fos, protein kinases such as PKC and PKA. Examples of other proteins which may be phosphorylated in an animal experiencing pain include receptors and ion channels such as the NMDA or AMPA receptors. Regardless of whether the measure is of transcription, translation or phosphorylation an animal can be said to be experiencing “pain” if one measures at least a 2 fold increase or decrease in any of these parameters compared to an animal not experiencing pain. An animal can be further said to be experiencing “pain” if there is a 3, 4, 5, 6, 7, 8, and up to 10 fold increase in the measurement of any of the above parameters compared to an animal not experiencing “pain”.

As used herein, “pain” refers to any of the behavioral, electrophysiological, or neurochemical criteria described above. In addition, “pain” can be assessed using combinations of these criteria.

As used herein, “pain” can refer to “pain” experienced by an animal as a result of accidental trauma (e.g., falling trauma, burn trauma, toxic trauma, etc.), congenital deformity or malformation, infection (e.g., inflammatory pain), or other conditions which are not within the control of the animal experiencing the “pain”. Alternatively, “pain” may be inflicted onto an animal by subjecting the animal to one or more “pain models”.

As used herein, “pain” can also be determined based on perception of pain by an individual (i.e., a patient). For example, mechanical pain may be assessed using a Pain Test Algometer (Wagner Instruments, Greenwich, Conn.), monofilament von Frey hairs, thermal pain by peltier or laser devices and pain may be scored in a human using known tests such as the visual analog scale which uses a 100 mm horizontal line marked with “no pain” on one end and “uncontrollable pain” on the other end, or a four-point verbal description based on a patients perception of no pain, mild, moderate, or severe pain. Other methods useful for determining the efficacy of pain treatment according to the invention include the peak B endorphin measurement assay (Neuroscience Toolworks, Inc., Evanston, Ill.), the human pain assays described by Fillingim et al. (2004, Anesthesiology 100:1263-1270) functional magnetic resonance imaging (fMRI), the brief pain inventory (BPI), and the McGill questionnaire.

The present invention comprises polynucleotide sequences that are differentially expressed in nerve injury pain models, including SNI, chronic constriction injury, and segmental nerve lesion, as well as inflammatory pain models. It is also within the scope of the present invention that the polynucleotides described herein as being differentially expressed in nerve injury, or neuropathic pain models may be also differentially expressed in other pain models known to those of skill in the art. It is also contemplated, that the pain models described herein, as well as others known to those of skill in the art, may be used to assay for agents that treat pain by decreasing the activity of the complement cascade, and/or to confirm the ability of a given agent to treat pain in an animal (e.g., decrease the level of pain perceived by the animal using a pain assay described herein).

As used herein, a “pain model” refers to any manipulation of an animal during which the animal experiences “pain”, as defined above. “Pain models” can be classified as those that test the sensitivity of normal animals to intense or noxious stimuli. These tests include responses to thermal, mechanical, or chemical stimuli. Thermal stimuli is usually hot (42 to 55° C.) and includes radiant heat to the tail (the tail flick test) radiant heat to the plantar surface of the hindpaw (the Hargreaves test, supra), the hotplate test, and immersion of the hindpaw or tail in hot water. Alternatively, thermal stimuli can be cold stimulus (15° to −10° C.), such as immersion in cold water, acetone evaporation or cold plate tests which may be used to test cold pain responsiveness using the thresholds discussed above. The end points are latency to response and the duration of the response as well as vocalization and licking the paw, place preference as described above. Mechanical Stimuli typically involves measurements of the threshold for eliciting a withdrawal reflex of the hindpaw to graded strength monofilament von Frey hairs wherein one can measure the force of the filament required to elicit a reflex. Alternatively, mechanical stimuli can be a sustained pressure stimulus to a paw (e.g., the Ugo Basila analgesiometer). The duration of response to a standard pin prick can also be measured. Threshold values for identifying a stimulus that causes “pain” to the animal are described above. Chemical Stimuli typically involves the application or injection of a chemical irritant to the skin, muscle joints or internal organs like the bladder or peritoneum. Irritants can include capsaicin, mustard oil, bradykinin, ATP, formalin, or acetic acid. The outcome measures include vocalization, licking the paw, writhing or spontaneous flexion.

Alternatively, a “pain model” can be a test that measures changes in the excitability of the peripheral or central components of the pain neural pathway pain sensitization, termed “peripheral sensitization” and “central sensitization”. “Peripheral Sensitization” involves changes in the threshold and responsiveness of the peripheral terminals of high threshold nociceptors which can be induced by: repeated heat stimuli, or application or injection of sensitizing chemicals (e.g. prostaglandins, bradykinin, histamine, serotonin, capsaicin, mustard oil). The outcome measures are thermal and mechanical sensitivity in the area of application/stimulation using the techniques described above in behaving animals or electrophysiological measurements of single sensory fiber receptive field properties either in vivo or using isolated skin nerve preparations. “Central sensitization” involves changes in the excitability of neurons in the central nervous system induced by activity in peripheral pain fibers. “Central sensitization” can be induced by noxious stimuli (e.g., heat) chemical irritants (e.g., injection/application of capsaicin/mustard oil or formalin or electrical activation of sensory fibers). The outcome measures are: behavioral, electrophysiological, and neurochemical.

Alternatively, a “pain model” can refer to those tests that measure the effect of peripheral inflammation on pain sensitivity. The inflammation can be produced by injection of an irritant such as complete Freunds adjuvant, carrageenan, turpentine, croton oil etc into the skin, subcutaneously, into a muscle into a joint or into a visceral organ. Production of a controlled UV light burn and ischemia can also be used. Administration of cytokines or inflammatory mediators such as lipopolysaccharide (LPS), or nerve growth factor (NGF) can mimic the effects of inflammation. The outcome of these models may also be measured as behavioral, electrophysiological, and/or neurochemical changes.

Further, a “pain model” includes those tests that mimic peripheral neuropathic pain using lesions of the peripheral nervous system. Examples of such lesions include, but are not limited to ligation of a spinal segmental nerve (CHUNG model; Kim and Chung, 1992, Pain, 50:355-63), partial nerve injury (Seltzer, 1979, Pain, 29: 1061), Spared Nerve Injury model (Decosterd and Woolf, 2000, Pain 87:149), chronic constriction injury (Bennett, 1993 Muscle Nerve 16: 1040), toxic neuropathies, such as diabetes (streptozocin model), pyridoxine neuropathy, taxol, vincristine and other antineoplastic agent-induced neuropathies, ischaemia to a nerve, peripheral neuritis models (e.g., CFA applied perineurally), models of postherpetic neuralgia using HSV infection. Such neuropathic pain models are also referred to herein as a “nerve injury pain model”. The outcome of these neuropathic or nerve injury “pain models” can be measured using behavioral, electrophysiological, and/or neurochemical criteria as described above.

In addition, a “pain model” refers to those tests that mimic central neuropathic pain using lesions of the central nervous system. For example, central neuropathic pain may be modeled by mechanical compressive, ischemic, infective, or chemical injury to the spinal cord of an animal. The outcome of such a model is measured using the behavioral, electrophysiological, and/or neurochemical criteria described above.

Activation of the Complement Cascade

The complement cascade is a group of proteins found in serum which work with antibody activity to eliminate pathogens in the body, a form of innate immunity. The complement cascade stimulates inflammation, facilitates antigen phagocytosis, and the lysis of some cells directly. The components of the complement cascade are well understood in the art, and are described, for example, in Walport, M. J. (2001, N. Engl. J. Med. 344: 1058-1066 and 1140-1144); Makrides, S. C. (1998, Pharmacological Reviews 50:59-78); Janeway et al. (1999, Immunobiology, Garland Publishing NY, N.Y.).

Table 1 shows components of the complement cascade and provides their Unigene ID number which can be used by one of skill in the art to readily access both nucleic acid and amino acid sequence information for each of the complement components shown. The Unigene sequence information can be used according to the invention to obtain antisense polynucleotides, double stranded RNA molecules, and antibodies specific for the components of the complement cascade. As shown in the table, Unigene reference numbers beginning with “Rn” represent rat sequence, and those beginning with “Hs” represent human sequences. The Unigene database is available on the world wide web at ncbi.nlm.nih.gov.

TABLE 1
Complement cascade

| | | |
|---|---|---|
| Unigene No. | Gene | Reference |

Complement cascade

| | | |
|---|---|---|
| Rn.105647 | C1q alpha | Reid, K. B., Biochem. J. 179 (2), 367-371 (1979) |
| Hs.9641 | | |
| Rn.6702 | C1q beta | Tissot et al., Biochemistry 44 (7), 2602-2609 (2005) |
| Hs.8986 | | |
| Rn.2393 | C1q gamma | Reid, K. B., Biochem. J. 179 (2), 367-371 (1979) |
| Hs.467753 | | |
| Rn.70397 | C1r | Nakagawa et al., Ann. Hum. Genet. 67 (PT 3), 207-215 |
| Hs.524224 | | (2003) |
| Rn.4037 | C1s | Kusumoto et al., Proc. Natl. Acad. Sci. U.S.A. 85 (19), 7307-7311 |
| Hs.458355 | | (1988) |
| Rn.2765 | C1q binding | Zhang et al., Immunology 115 (1), 63-73 (2005) |
| Hs.97199 | protein | |
| Rn.98333 | C2 | Bentley, Proc. Natl. Acad. Sci. U.S.A. 81 (4), 1212-1215 |
| Hs.408903 | | (1984) |
| Rn.81052 | C4, C4a, C4b | Teisberg et al., Nature 264 (5583), 253-254 (1976) |
| Hs.546241 | | |
| Rn.9667 | Mb12 | Sastry et al., J. Exp. Med. 170 (4), 1175-1189 (1989) |
| Hs.499674 | | |
| Rn.49256 | Masp1 | Takada et al., Biochem. Biophys. Res. Commun. 196 (2), |
| Hs.89983 | | 1003-1009 (1993) |
| Rn.45144 | Masp2 | Thiel et al., Nature 386 (6624), 506-510 (1997) |
| Hs.119983 | | |
| Rn.109148 | bf, properdin | Cislo et al., Immunol. Lett. 80 (3), 145-149 (2002) |
| Hs.69771 | | |
| Rn.16172 | Adn | Niemann et al., Biochemistry 23 (11), 2482-2486 (1984) |
| Hs.155597 | | |
| Rn.11378 | C3, C3a, C3b | de Bruijn, Proc. Natl. Acad. Sci. U.S.A. 82 (3), 708-712 |
| Hs.529053 | | (1985) |
| Rn.9772 | C3ar1 | Crass et al., Eur. J. Immunol. 26 (8), 1944-1950 (1996) |
| Hs.527839 | | |
| Rn.23009 | C5, C5a, C5b | Haviland et al., J. Immunol. 146 (1), 362-368 (1991) |
| Hs.494997 | | |
| Rn.10680 | C5r1 | Gerard et al., Biochemistry 32 (5), 1243-1250 (1993) |
| Hs.2161 | | |
| Rn.16145 | C6 | Haefliger et al., J. Biol. Chem. 264 (30), 18041-18051 (1989) |
| Hs.481992 | | |
| Rn.139495 | C7 | Hobart et al., J. Immunol. 154 (10), 5188-5194 (1995) |
| Hs.78065 | | |
| Rn.110603 | C8b | Howard et al., Biochemistry 26 (12), 3565-3570 (1987) |
| Hs.391835 | | |
| Hs.93210 | C8a | Rao et al., Biochemistry 26 (12), 3556-3564 (1987) |
| Rn.10252 | C9 | Stanley et al., EMBO J. 4 (2), 375-382 (1985) |
| Hs.1290 | | |

Complement regulators

| | | |
|---|---|---|
| Rn.100285 | C1 inhibitor | Tosi et al., Gene 42 (3), 265-272 (1986) |
| Hs.384598 | | |
| Rn.10408 | C4bpa | Rodriguez de Cordoba et al., J. Exp. Med. 173 (5), 1073-1082 |
| Hs.1012 | | (1991) |
| Rn.11151 | C4bp-ps1 | Hillarp et al., J. Biol. Chem. 268 (20), 15017-15023 (1993) |
| Hs.99886 | | |
| Rn.101777 | Cfh | Skerka et al., J. Biol. Chem. 272 (9), 5627-5634 (1997) |
| Hs.553515 | | |
| Rn.7424 | Cfi | Catterall et al., Biochem. J. 242 (3), 849-856 (1987) |
| Hs.312485 | | |
| Rn.87493 | Vtn | Suzuki et al., EMBO J. 4 (10), 2519-2524 (1985) |
| Hs.2257 | | |
| Rn.5825 | Crry | Quigg et al., Immunogenetics 42 (5), 362-367 (1995) |
| Rn.18841 | Daf1 | Caras et al., Nature 325 (6104), 545-549 (1987) |
| Hs.527653 | | |
| Rn.73851 | mcp | Cervoni et al., Mol. Reprod. Dev. 34 (1), 107-113 (1993) |
| Hs.510402 | | |
| Rn.1231 | Cd59 | Davies et al., J. Exp. Med. 170 (3), 637-654 (1989) |
| Hs.278573 | | |
| Rn.8937 | S100b | Rustandi et al., Nat. Struct. Biol. 7, 575 (2000) |

Methods for determining the activation of the complement cascade (e.g., by assaying for the activity of components of the cascade or by assaying for the activity of the ultimate effector molecules of the cascade) are known in the art, and may be found, for example, in U.S. Pat. Nos. 6,750,334; 5,711,959; 5,348,876, and 6,586,559. For example, the total hemolytic complement assay (CH50) measures the ability of the classical pathway and the membrane attack complex (MAC) to lyse a sheep RBC to which an antibody has been attached. The alternative pathway CH50 (rabbit CH50 or APCH50) measures the ability of the alternative pathway and the MAC to lyse a rabbit RBC. Hemolytic assays can be used to measure functional activity of specific components of either pathway. Complement proteins can also be measured using antigenic techniques (e.g., nephelometry, agar gel diffusion, radial immunodiffusion). Complement activation may be determined by hemolytic assays known in the art and described, for example, in U.S. Pat. No. 5,098,977.

In an alternate assay for complement activation, a cell which expresses a particular antigen on its surface is loaded with a detectable substance, e.g., a fluorescent dye, and then contacted with an surface antigen-specific immunoglobulin and a complement source (e.g., purified guinea pig complement or human serum as a source of human complement). Cell lysis, as determined by release of the fluorescent dye from the cells, is determined as an indication of activation of the complement cascade upon binding of the immunoglobulin to the antigen on the cell surface. Cells which do not express the particular antigen on their surface are used as a negative control.

In another complement activation assay, the ability of a particular immunoglobulin to bind the first component of the complement cascade, C1q, can be assessed. For example, C1q binding can be determined using a solid phase assay in which ¹²⁵I-labeled human C1q is added to an amount of immunoglobulin with a its specific antigen, and the amount of bound ¹²⁵I-labeled human C1q quantitated. C1q binding assays are described further in Tan. L. K., et al. (1990) Proc. Natl. Acad. Sci. USA 87:162-166; and Duncan, A. R. and G. Winter (1988) Nature 332:738-740.

Assays specific for the activation and/or activity of particular components of the complement cascade are also known in the art (See, e.g., Wagner and Hugli, 1984, Anal. Biochem. 136: 75-88 (teaches radioimmunoassays for complement components C3a, C4a, and C5a, which is also indicative of the activation of components C3, C4, and C5); Adelsberg et al., 1985, Diagn. Immunol. 3: 187-190 (teaches quantitative assays for the complement component C3d in plasma); Caporale et al., 2000, J. Biol. Chem. 275:378-385 (teaches assays for the activation of complement components CVFBb, C4b2a, and C1s); Linder et al., 1981, J. Immunol. Methods 47:49-59 (teaches assays for the activation of complement components C1q, C3 and C4); Petersen et al., 2001, J. Immunol. Methods 257:107-116 (teaches an assay for determining the activation of the mannan-binding lectin pathway of complement activation); Mayes et al., 1984, J. Clin. Invest. 73:160-170 (teaches ELISA assays for determining the activation of the third component of complement (C3b), proteolytic fragment of complement Factor B (Bb), and properdin (P) complex or its derivative product, C3b,P); and Sohn et al., 2000, Ivest. Ophthalmol. Vis. Sci. 41:3492-3502 (teaches assays for the membrane attack complex, C3 activation products)). In addition, assays for complement activation are available from a number of commercial vendors (e.g., Amersham Biosciences/GE Healthcare, Sigma-Aldrich; and Merck).

Additional assays for complement activation have been described in the art and are known to the skilled artisan.

Inhibition of Complement Activation

The present invention provides a method for the treatment of pain by administering to an animal an agent that decreases the activity of the complement cascade or the availability of its components. Preferably, and agent useful in the invention results in a decrease in the activity of the complement cascade of at least 10% or more, including 20%, 30%, 40%, 50%, 60%, 70%, 80%, 90%, and up to 100% or more, in response to an agent relative to the activity of the cascade in the absence of the agent. The activity of the complement cascade refers to the activity of the individual components of the cascade (for example, the activity of C3b is to form part of the C5 convertase, and to bind to cells making them more susceptible to phagocytosis), and also refers to the activity of the final effector molecules of the cascade (e.g., activity of the C5b6789 membrane attack complex to either cause osmotic lysis of a cell or to allow ion flux across the cell membrane). The activity of the complement cascade, and of the individual components of the complement cascade is known in the art, and may be found, for example, in Makrides, S. C. (1998, Pharmacological Reviews 50:59-78) and Janeway et al. (1999, Immunobiology, Garland Publishing NY, N.Y.). An agent that decreases the activity of the complement cascade refers to a protein, antibody, enzyme, small molecule, antisense RNA, or siRNA that may decrease the activity of a component of the complement cascade, and/or decreases the activity of the ultimate effector molecules of the complement cascade by at least 10% or more, including 20%, 30%, 40%, 50%, 60%, 70%, 80%, 90%, and up to 100% or more relative to the activity of the complement cascade in the absence of the agent. An agent that decreases the activity of the complement cascade can also include an agent which prevents the assembly of a final effector molecule of the cascade, such as the membrane attack complex. Thus, an antibody, for example, which binds to complement component C6 would be useful to block the assembly of the membrane attack complex and decrease the activity of the complement cascade.

An agent or therapeutic agent according to the invention can ameliorate at least one of the symptoms and/or physiological changes associated with pain including, but not limited to mechanical allodynia and hyperalgesia, and temperature allodynia and hyperalgesia.

The candidate therapeutic agent may be a synthetic compound, or a mixture of compounds, or may be a natural product (e.g. a plant extract or culture supernatant). According to the invention, a therapeutic agent or compound can be a candidate or test compound. Similarly, according to the invention, a candidate or test compound can be a therapeutic agent.

An agent that decreases the activity of the complement cascade is preferably an antibody polypeptide that specifically binds to a component of the complement cascade, an antisense oligonucleotide that inhibits the expression of a polynucleotide sequence encoding a component of the complement cascade, a double stranded RNA molecule, or a compound including, but not limited to the following compounds: soluble complement receptor type 1, soluble complement receptor type 1 lacking long homologous repeat-A, soluble complement receptor type 1-sialyl lewis, complement receptor type 2, soluble decay accelerating factor, soluble membrane cofactor protein, soluble CD59, decay accelerating factor-CD59 hybrid, membrane cofactor protein-decay accelerating factor hybrid, C1 inhibitor, C1q receptor, C089, PR226, CBP2, DFP, BCX-1470, TKIXc, K-76 COOH, FUT-175, PS-oligo, Glycyrrhizin, GR-2II, AGIIb-1, AR-2IIa, Rosmarinic acid, BR-5-I, Fucan, complestatin, decorin, dextran, heparin, LU51198, GCRF, CSPG, C4 inactivator, compstatin, CR1 (CD35), CD2 (CD21), MCP (CD46), DAF (CD55), factor H, C3BP, Crry, TP-10, plasma-derived protein C1 esterase inhibitor, vaccinia virus complement control protein, AcF[OPdCHaWR], CGS32359, 3D53, SB-290157, and cobra venom factor. Specific agents that decrease the activation of the complement cascade are known in the art and may be found, for example, in Holland et al. (2004, Curr. Opin. Investig. Drugs 5: 1164-1173), Makrides (1998, Pharma. Reviews 50: 59-78), Mollnes and Kirschfink (2005, Molecular Immunology 43:107-121), and Hart et al. (2004, Mol. Immunology 41: 165-141).

Specific agents that may be used to decrease the activation of the complement cascade include Naturally occurring complement regulators such as C1-inhibitor, regulators of complement activation (CR1 (CD35), CR2 (CD21), MCP (CD46), DAF (CD55), factor H and C4BP), Crry, soluble CR1, soluble DAF and MCP, and soluble CD59.

In addition to blocking the activity of specific components of the complement cascade, the present invention also contemplates that the activity of the complement cascade can be decreased by blocking receptors for the components of the cascade. For example, the complement components C3a and C5a bind to G-protein coupled receptors. The activity of the complement cascade could be decreased, therefore, by antagonizing the binding of either of these components to their respective receptors. C3a and C5a receptor antagonists include, but are not limited to the C5aR mAb 20/70, C3-binding peptide compstatin, 3D53 (a synthetic peptidic antagonist of the C5a anaphylatoxin receptor), SB-290157 (non-peptidergic antagonist of the C3a anaphylatoxin receptor), and AcF-[OpdChaWR]. Other receptor antagonists are known in the art and are described, for example, in March et al., Mol Pharmacol. April 2004;65(4):868-79; Holland et al., Curr Opin Investig Drugs. November 2004;5(11):1164-73; Wong et al., IDrugs. July 1999; 2(7):686-93; Buck and Wells, Proc Natl Acad Sci USA. Feb. 22, 2005;102(8):2719-24. Epub Feb. 14, 2005; Higginbottom et al., J Biol Chem. May 6, 2005;280(18):17831-40. Epub Jan 20, 2005; and Allegretti et al., Curr Med Chem. 2005;12(2):217-36.

Small Molecules

Useful agents for decreasing the activity of complement may be found within numerous chemical classes. Useful compounds may be organic compounds, or small organic compounds. Small organic compounds, or “small molecules” have a molecular weight of more than 50 yet less than about 2,500 daltons, preferably less than about 750, more preferably less than about 350 daltons. Exemplary classes include heterocycles, peptides, saccharides, steroids, and the like. Small molecules can be nucleic acids, peptides, polypeptides, peptidomimetics, carbohydrates, lipids or other organic (carbon-containing) or inorganic molecules. The compounds may be modified to enhance efficacy, stability, pharmaceutical compatibility, and the like. Structural identification of an agent may be used to identify, generate, or screen additional agents. For example, where peptide agents are identified, they may be modified in a variety of ways to enhance their stability, such as using an unnatural amino acid, such as a D-amino acid, particularly D-alanine, by functionalizing the amino or carboxylic terminus, e.g. for the amino group, acylation or alkylation, and for the carboxyl group, esterification or amidification, or the like.

Small molecules that may be used to decrease the activity of the complement cascade include, but are not limited to C1 binding peptides (see, e.g., Lauvrak et al., 1997, Biol. Chem. 378: 1509-1519; Roos et al., 2001, J. Immunol. 167: 7052-7059), compstatin (Morikis et al., 1998, Protein Sci. 7:619-627), C3aR antagonists (e.g., SB290157; Ames et al., 2001 J. Immunol. 166:6341-6348), and C5aR antagonists (e.g., AcF[OPdChaWR]; Fitch et al., 1999, Circulation 100:2499-2506).

Antisense Therapy

In one embodiment, a therapeutic agent, according to the invention, can be a nucleic acid sequence encoding a component of the complement cascade or a sequence complementary thereto, useful in antisense therapy. The antisense sequence of a polynucletoide encoding a component of the complement cascade may be determined using the sequence indicated by Universal Identifier in Table 1. As used herein, antisense therapy refers to administration or in situ generation of oligonucleotide molecules or their derivatives which specifically hybridize (e.g., bind) under cellular conditions with the cellular mRNA and/or genomic DNA, thereby inhibiting transcription and/or translation of that gene. The binding may be by conventional base pair complementarity, or, for example, in the case of binding to DNA duplexes, through specific interactions in the major groove of the double helix. In general, antisense therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the cellular mRNA identified as being differentially expressed in an animal subjected to pain. The construction and use of expression plasmids is described above and may be adapted by one of skill in the art to include expression plasmids or vectors comprising antisense oligonucleotides. Alternatively, the antisense construct is an oligonucleotide probe which is generated ex vivo and which, when introduced into the cell, causes inhibition of expression by hybridizing with the mRNA and/or genomic sequences of a differentially expressed nucleic acid. Such oligonucleotide probes are preferably modified oligonucleotides which are resistant to endogenous nucleases, e.g., exonucleases and/or endonucleases, and are therefore stable in vivo. Exemplary nucleic acid molecules for use as antisense oligonucleotides are phosphoramidate, phosphorothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668. With respect to antisense DNA, oligodeoxyribonucleotides derived from the translation initiation site, e.g., between the −10 and +10 regions of the nucleotide sequence of interest, are preferred.

Antisense approaches involve the design of oligonucleotides (either DNA or RNA) that are complementary to mRNA (i.e., differentially expressed mRNA). The antisense oligonucleotides will bind to the mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of mRNA encoding a complement component, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. (Wagner, R. 1994. Nature 372:333). Therefore, oligonucleotides complementary to either the 5′ or 3′ untranslated, non-coding regions of a gene could be used in an antisense approach to inhibit translation of endogenous mRNA. Oligonucleotides complementary to the 5′ untranslated region of the mRNA should include the complement of the AUG start codon. Antisense oligonucleotides complementary to mRNA coding regions are typically less efficient inhibitors of translation but could also be used in accordance with the invention. Whether designed to hybridize to the 5′, 3′, or coding region of subject mRNA, antisense nucleic acids should be at least six nucleotides in length, and are preferably less than about 100 and more preferably less than about 50, 25, 17 or 10 nucleotides in length.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO 88/098 10, published Dec. 15, 1988) or the blood-brain barrier (see, e.g., PCT Publication No. WO 89/10 134, published Apr. 25, 1988), hybridization-triggered cleavage agents (See, e.g., Krol et al., 1988, BioTechniques 6:958-976), or intercalating agents (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytriethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Peny-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methyiphosphonate, an alkyl phosphotriester, and a formacetal or analog thereof.

In yet a further embodiment, the antisense oligonucleotide is an α-anomeric oligonucleotide. An α-anomeric oligonucleotide forms specific double-stranded hybrids with complementary RNA in which, contrary to the usual n-units, the strands run parallel to each other (Gautier et al, 1987, Nucl. Acids Res. 15:6625-6641). The oligonucleotide is a 2′-O-methylribonucleotide (Inoue et al., 1987, Nucl. Acids Res. 15:6131-12148), or a chimeric RNA-DNA analogue (Jnoue et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.) based on the known sequence of the differentially expressed nucleic acid sequences. As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

While antisense nucleotides complementary to a coding region sequence can be used, those complementary to the transcribed untranslated region and to the region comprising the initiating methionine are most preferred.

The antisense molecules can be delivered to cells which express the target nucleic acid in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation on endogenous mRNAs. Therefore, a preferred approach utilizes a recombinant DNA construct in which the antisense oligonucleotide is placed under the control of a strong pol III or pol II promoter. The use of such a construct to transfect target cells in an animal will result in the transcription of sufficient amounts of single stranded RNAs that will form complementary base pairs with the endogenous transcripts and thereby prevent translation of the target mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art, combined with those described above. Vectors can be plasmid, viral, or others known in the art for replication and expression in mammalian cells. Expression of the sequence encoding the antisense RNA can be by any promoter known in the art to act in animal, preferably mammalian cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et at, 1982, Nature 296:39-42), etc. Any type of plasmid, cosmid, YAC or viral vector can be used to prepare the recombinant DNA construct which can be introduced directly into the tissue site; e.g., the spinal cord, or dorsal root ganglion. Alternatively, viral vectors can be used which selectively infect the desired tissue (e.g., for brain, herpesvirus vectors may be used), in which case administration may be accomplished by another route (e.g., systemically).

Ribozymes

In another aspect of the invention, ribozyme molecules designed to catalytically cleave target mRNA transcripts can be used to prevent translation of target mRNA and expression of a target protein (See, e.g., PCT International Publication WO90/11364, published Oct. 4, 1990; Sarver et al., 1990, Science 247:1222-1225 and U.S. Pat. No. 5,093,246). While ribozymes that cleave mRNA at site specific recognition sequences can be used to destroy target mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. Ribozymes, useful in the present invention may be designed based on the known sequence of the nucleic acid sequence identified as being differentially expressed in an animal subjected to pain as described above. The construction and production of hammerhead ribozymes is well known in the art and is described more fully in Haseloff and Gerlach, 1988, Nature, 334:585-591. Preferably the ribozyme is engineered so that the cleavage recognition site is located near the 5′ end of the target mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO88/04300 by University Patents Inc.; Been and Cech, 1986, Cell, 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in a target gene.

As in the antisense approach, the ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous messages and inhibit translation. Because ribozymes, unlike antisense molecules, are catalytic, a lower intracellular concentration is required for efficiency.

Antisense RNA, DNA, and ribozyme molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. The sequences of the antisense and ribozyme molecules will be based on the known sequence of the differentially expressed nucleic acid molecules. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ 0-methyl rather than phosphodiesterase linkages within the oligodeoxyribonucleotide backbone.

RNAi Therapy

In another embodiment, a therapeutic agent according to the invention can be a double stranded RNAi molecule that is specifically targeted to a polynucleotide sequence that encodes a component of the complement cascade. As used herein, RNAi or RNA interference refers to the gene-specific, double stranded RNA (dsRNA) mediated, post-transcriptional silencing of gene expression as described in the review by Hannon, G., (2002) Nature 418, 244-250, which is herein incorporated in its entirety. Current experimental evidence indicates that RNA is specific for a target RNA are recognized and processed into 21 and 23 nucleotide small interfering RNAs (siRNAs) by the Dicer RNase III endonuclease. SiRNAs are then incorporated into a RNA induced silencing complex (RISC) which becomes activated by unwinding of the duplex siRNA. Activated RISC complexes then promote RNA degradation and translation inhibition of the target RNA.

In mammals, RNAi therapy, according to the invention, refers to gene-specific suppression that can be achieved by generating siRNA (Elbashir, S. M. et al. (2001) Nature (London) 411, 494-498). In vitro synthesized siRNAs can be prepared by any method known in the art for the synthesis of RNA molecules. These include techniques for chemically synthesizing oligoribonucleotides that are well known in the art, for example, solid phase phosphoramidite chemical synthesis. The sequences of the siRNA molecules are based on the known sequence of the differentially expressed nucleic acid molecules. Alternatively, siRNA molecules can be generated by the T7 or SP6 polymerase promoter driven in vitro transcription of DNA sequences encoding the siRNA molecule. In vitro synthesized siRNAs can be delivered to cells either by direct injection of in vitro synthesized siRNAs into the tissue site. Alternatively, modified siRNAs, designed to target the desired cells (via linkage to peptides or antibodies that specifically bind to cell surface receptors or antigens), can be administered systemically.

In a preferred embodiment, the siRNAs of the invention are delivered to a target cell as an expression plasmid under the control of a RNA polymerase II or III promoter. When transcribed in the cell, siRNA is generated which is complementary to a cellular mRNA identified as being differentially expressed in an animal subjected to pain. The construction and use of expression plasmids is described above and may be adapted by one of skill in the art to include siRNA expression plasmids. Such vectors can be constructed by recombinant DNA technology methods standard in the art, combined with those described above. Vectors can be plasmid, viral, or others known in the art for replication and expression in mammalian cells. Expression of the sequence encoding the siRNA can be by any promoter known in the art to act in an animal, preferably mammalian cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et at, 1982, Nature 296:39-42), etc as well as neural specific promoters, for example the nestin promoter. Any plasmid, cosmid, YAC or viral vector can be used to prepare the recombinant DNA construct which can be introduced directly into the tissue site; e.g., the spinal cord, or dorsal root ganglion. Alternatively, viral vectors can be used which selectively infect the desired tissue (e.g., for brain, herpes virus vectors may be used), in which case administration may be accomplished by another route (e.g., systemically).

In a preferred embodiment, the siRNA expression vectors of the invention are synthesized from a DNA template under the control of an RNA polymerase III (Pol III) promoter in transfected cells or transgenic animals (see below). Pol III directs the synthesis of small, noncoding transcripts whose 3′ ends are defined by termination within a stretch of 4-5 thymidines (Ts) (Sui et al. PNAS (2002) vol. 99, 5515-5520). Addition of 3′ overhangs contributes to the activity of siRNA synthesized in vitro (Elbashir, S. M et al. (2001) Genes Dev. 15, 188-200). Transfection of such a construct into target cells results in the transcription of sufficient amounts of siRNAs to base pair with the endogenous transcripts, promote its degradation and thereby prevent translation of the target mRNA. The vector can remain episomal or become chromosomally integrated. Alternatively the construct may be incorporated into a viral vector such as herpes virus vectors as described supra.

An example of mouse U6 pol III transcribed siRNA expression plasmid is shown below where the 21 nucleotide sequence is specific for a polynucleotide sequence encoding a component of the complement cascade (see Sui et al. PNAS (2002) vol. 99, 5515-5520):

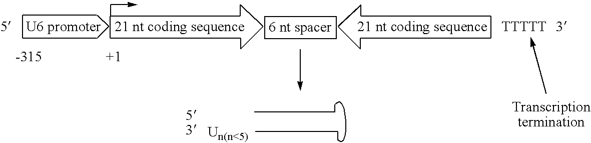

General guidelines for the selection of suitable RNAi target sequences are known in the art and include the following (outlined on the world wide web at rnaiweb.com):
1 .Targeted regions on the cDNA sequence of a targeted gene should be located 50-100 nt downstream of the start codon (ATG).
2. Search for sequence motif AA(N₁₉)TT or NA(N₂₁), or NAR(N₁₇)YNN, where N is any nucleotide, R is purine (A, G) and Y is pyrimidine (C, U).
3. Avoid targeting introns, since RNAi only works in the cytoplasm and not within the nucleus.
4. Avoid sequences with >50% G+C content.
5. Avoid stretches of 4 or more nucleotide repeats.
6. Avoid 5URT and 3UTR, although siRNAs targeting UTRs have successfully induced gene inhibition.
7. Avoid sequences that share a certain degree of homology with other related or unrelated genes.

Examples of target sequences for RNAi which may be used according to the invention are shown in the following table.

TABLE 2

| | |
|---|---|
| Unigene | Complement |

| | | |
|---|---|---|
| Ref. | component | Exemplary siRNA sequences |

| | | |
|---|---|---|
| Rn.6702 | C1q beta | atatctccca ggcccagctc ag |
| Hs.8986 | | ggcccagctc agctgcaccg gg |
| | | agctgcaccg ggcccccagc ca |
| | | ggcccccagc catccctggc at |
| Rn.70397 | C1r | ttctgtgggc aactgggttc tc |
| Hs.524224 | | tgtgggc aactgggttc tccac |
| | | gggc aactgggttc tccactgg |
| | | c aactgggttc tccactgggc a |
| Rn.2765 | C1q binding | gcctgctaca cggcccactc gg |
| Hs.97199 | protein | tgctaca cggcccactc gggca |
| | | taca cggcccactc gggcaagc |
| | | a cggcccactc gggcaagctg a |
| Rn.98333 | C2 | aatatctcgg gtggcacctt ca |
| Hs.408903 | | atctcgg gtggcacctt caccc |
| | | tcgg gtggcacctt caccctca |
| | | g gtggcacctt caccctcagc c |
| Rn.81052 | C4, C4a, C4b | tcatctg ggggtccccc ta |
| Hs.546241 | | tctg ggggtccccc tatcg |
| | | g ggggtccccc tatcggtg |
| | | ggtccccc tatcggtggg g |
| Rn.45144 | Masp2 | ctgagctcgg gggccaaggt gc |
| Hs.119983 | | agctcgg gggccaaggt gctgg |
| | | tcgg gggccaaggt gctggcca |
| | | g gggccaaggt gctggccacg c |
| Rn.109148 | bf, properdin | ctccaagagg gccaggcact gg |
| Hs.69771 | | caagagg gccaggcact ggagt |
| | | gagg gccaggcact ggagtacg |
| | | g gccaggcact ggagtacgtg t |
| Rn.16172 | Adn | gcagttctggtcctcctaggag |
| Hs.155597 | | gttctggtcctcctaggagcgg |
| | | ctggtcctcctaggagcggccg |
| | | gtcctcctaggagcggccgcct |
| Rn.11378 | C3, C3a, C3b | gcaaaaaact agtgctgtcc ag |
| Hs.529053 | | aaaaact agtgctgtcc agtga |
| | | aact agtgctgtcc agtgagaa |
| | | t agtgctgtcc agtgagaaga c |
| Rn.9772 | C3ar1 | actgtggcta agtgtgggga cc |
| Hs.527839 | | gtggcta agtgtgggga ccaga |
| | | gcta agtgtgggga ccagacag |
| | | a agtgtgggga ccagacagga c |
| Rn.23009 | C5, C5a, C5b | atttagttac tcctcaggcc at |
| Hs.494997 | | tagttac tcctcaggcc atgtt |
| | | ttac tcctcaggcc atgttcat |
| | | c tcctcaggcc atgttcattt a |
| Rn.10680 | C5r1 | atgaactccttcaattatacca |
| Hs.2161 | | aactccttcaattataccaccc |
| | | tccttcaattataccacccctg |
| | | ttcaattataccacccctgatt |
| Rn.16145 | C6 | tcaaaaactt gcaattctgg aa |
| Hs.481992 | | aaaactt gcaattctgg aaccc |
| | | actt gcaattctgg aacccaga |
| | | t gcaattctgg aacccagagc a |
| Rn.139495 | C7 | atgaaggtga taagcttatt ca |
| Hs.78065 | | aaggtga taagcttatt cattt |
| | | gtga taagcttatt cattttgg |
| | | a taagcttatt cattttggtg g |
| Rn.110603 | C8b | ggcactcaca gcacaggctt gt |
| Hs.391835 | | actcaca gcacaggctt gttat |
| | | caca gcacaggctt gttatggg |
| | | a gcacaggctt gttatgggtc t |
| Hs.93210 | C8a | tttttttttt catcctactt tg |
| | | ttttttt catcctactt tgttt |
| | | tttt catcctactt tgttttat |
| | | t catcctactt tgttttattg g |
| Rn.10252 | C9 | cagcatgtca gcctgccgga gc |
| Hs.1290 | | catgtca gcctgccgga gcttt |
| | | gtca gcctgccgga gctttgca |
| | | a gcctgccgga gctttgcagt t |
| Rn.100285 | C1 inhibitor | ctgatttaca ggaactcaca cc |
| Hs.384598 | | atttaca ggaactcaca ccagc |
| | | taca ggaactcaca ccagcgat |
| | | a ggaactcaca ccagcgatca a |
| Rn.10408 | C4bpa | aaaactctga tctggggagg aa |
| Hs.1012 | | actctga tctggggagg aacca |
| | | ctga tctggggagg aaccagga |
| | | a tctggggagg aaccaggact a |
| Rn.11151 | C4bp-ps1 | attctgtctt tcacatacat tg |
| Hs.99886 | | ctgtctt tcacatacat tgaga |
| | | tctt tcacatacat tgagacca |
| | | t tcacatacat tgagaccaaa a |
| Rn.101777 | Cfh | ggaattcggg cacgagtgaa ag |
| Hs.553515 | | attcggg cacgagtgaa agatt |
| | | cggg cacgagtgaa agatttca |
| | | g cacgagtgaa agatttcaaa c |
| Rn.7424 | Cfi | cgaacacctc caacatgaag ct |
| Hs.312485 | | acacctc caacatgaag cttct |
| | | cctc caacatgaag cttcttca |
| | | c caacatgaag cttcttcatg t |
| Rn.87493 | Vtn | caatcatgga tcaatagcta tg |
| Hs.2257 | | tcatgga tcaatagcta tgttt |
| | | tgga tcaatagcta tgtttgga |
| | | a tcaatagcta tgtttggaga a |
| Rn.5825 | Crry | acactctggg cgcggagcac aa |
| | | ctctggg cgcggagcac aatga |
| | | tggg cgcggagcac aatgattg |
| | | g cgcggagcac aatgattggt c |
| Rn.18841 | Daf1 | cccggggcgt atgacgccgg ag |
| Hs.527653 | | ggggcgt atgacgccgg agccc |
| | | gcgt atgacgccgg agccctct |
| | | t atgacgccgg agccctctga c |
| Rn.1231 | Cd59 | gggccggggg gcggagcctt gc |
| Hs.278573 | | ccggggg gcggagcctt gcggg |
| | | gggg gcggagcctt gcgggctg |
| | | g gcggagcctt gcgggctgga g |
| Rn.8937 | S100b | cttttatctc ttaggaaatc aa |
| | | ttatctc ttaggaaatc aaaga |
| | | tctc ttaggaaatc aaagagca |
| | | c ttaggaaatc aaagagcagg a |

Antibody Polypeptides

The present invention also provides antibody polypeptides that are specifically immunoreactive to components of the complement cascade as described above. The antibody polypeptides may be polyclonal or monoclonal or recombinant, produced by methods known in the art or as described below.

As use herein, the term “specifically immunoreactive” refers to a measurable and reproducible specific immunoreaction such as binding between a peptide and an antibody, that is determinative of the presence of the peptide in the presence of a heterogeneous population of peptides and other biologics. The term “specifically immunoreactive” may include specific recognition of structural shapes and surface features. Thus, under designated conditions, an antibody specifically immunoreactive to a particular peptide does not bind in a significant amount to other peptides present in the sample. An antibody specifically immunoreactive to a peptide has an association constant of at least 10³M⁻¹ or 10⁴M⁻¹, sometimes about 10⁵M⁻¹ or 10⁶M⁻¹, in other instances 10⁶M⁻¹ or 10⁷M⁻¹, preferably about 10⁸M⁻¹ to 10⁹M⁻¹, and more preferably, about 10¹⁰M⁻¹ to 10¹¹M⁻¹ or higher. A variety of immunoassay formats can be used to determine antibodies specifically immunoreactive to a particular peptide. For example, solid-phase ELISA immunoassays are routinely used to select monoclonal antibodies specifically immunoreactive with a peptide. See, e.g., Harlow and Lane (1988) Antibodies, A Laboratory Manual, Cold Spring Harbor Publications, New York, for a description of immunoassay formats and conditions that can be used to determine specific immunoreactivity.

An antibody polypeptide includes a polypeptide which either is an antibody or is a part of an antibody, modified or unmodified, which retains the ability to specifically bind antigen. Thus, the antibody polypeptides include whole antibody, an antigen-binding heavy chain, light chain, heavy chain-light chain dimer, Fab fragment, F(ab′)2 fragment, dAb, or an Fv fragment, including a single chain Fv (scFv). The phrase “antibody polypeptide” is intended to encompass recombinant fusion polypeptides that comprise an antibody polypeptide sequence that retains the ability to specifically bind antigen in the context of the fusion. Antibody polypeptides may be labeled with detectable labels by one of skill in the art. The label can be a radioisotope, fluorescent compound, chemiluminescent compound, enzyme, or enzyme co-factor, or any other labels known in the art. In some aspects, the antibody that binds to an entity one wishes to measure (the primary antibody) is not labeled, but is instead detected by binding of a labeled secondary antibody that specifically binds to the primary antibody.

Antibody polypeptides of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), intracellularly made antibodies (i.e., intrabodies), and epitope-binding fragments of any of the above. The antibodies of the invention can be from any animal origin including birds and mammals. Preferably, the antibody polypeptides are of human, murine (e.g., mouse and rat), donkey, sheep, rabbit, goat, guinea pig, camel, horse, or chicken origin.

As used herein, a “monoclonal antibody” refers to an antibody polypeptide that recognizes only one type of antigen. This type of antibody polypeptide is produced by the daughter cells of a single antibody-producing hybridoma. A monoclonal antibody typically displays a single binding affinity for any epitope with which it immunoreacts. A monoclonal antibody may contain an antibody molecule having a plurality of antibody combining sites, each immunospecific for a different epitope, e.g., a bispecific monoclonal antibody. Monoclonal antibodies may be obtained by methods known to those skilled in the art. (Kohler and Milstein (1975), Nature, 256:495-497; U.S. Pat. No. 4,376,110; Ausubel et al. (1987, 1992), eds., Current Protocols in Molecular Biology, Greene Publishing Assoc. and Wiley Interscience, N.Y.; Harlow and Lane (1988), ANTIBODIES: A Laboratory Manual, Cold Spring Harbor Laboratory; Colligan et al. (1992, 1993), eds., Current Protocols in Immunology, Greene Publishing Assoc. and Wiley Interscience, N.Y.).

The antibodies of the present invention can be monospecific or multispecific (e.g., bispecific, trispecific, or of greater multispecificity). Multispecific antibodies can be specific for different epitopes of a component of the complement cascade, or can be specific for both a component of the complement cascade, and a heterologous epitope, such as a heterologous peptide or solid support material. (See, e.g., WO 93/17715; WO 92/08802; WO 91/00360; WO 92/05793; Tutt et al., 1991, J. Immunol., 147:60-69; U.S. Pat. Nos. 4,474,893; 4,714,681; 4,925,648; 5,573,920; 5,601,819; and Kostelny et al., 1992, J. Immunol., 148:1547-1553).

Moreover, antibodies can also be prepared against any region of the complement cascade components. In addition, if a polypeptide is a membrane protein, e.g., a receptor protein, antibodies can be developed against the entire receptor or epitope of the receptor comprising at least 6 amino acid residues, for example, an intracellular domain, an extracellular domain, the entire transmembrane domain, specific transmembrane segments, any of the intracellular or extracellular loops, or any portions of these regions. Antibodies can also be developed against specific functional sites, such as the site of ligand binding, or sites that are glycosylated, phosphorylated, myristylated, or amidated, for example.

In the present invention, the components of the complement cascade used for generating antibodies preferably contain a sequence of at least 4, at least 5, at least 6, at least 7, more preferably at least 8, at least 9, at least 10, at least 11, at least 12, at least 13, at least 14, at least 15, and, preferably, between about 5 to about 50 amino acids in length, more preferably between about 10 to about 30 amino acids in length, even more preferably between about 10 to about 20 amino acids in length.

The monoclonal antibodies of the present invention can be prepared using well-established methods. In one embodiment, the monoclonal antibodies are prepared using hybridoma technology, such as those described by Kohler and Milstein (1975, Nature, 256:495) and Goding (Monoclonal Antibodies: Principles and Practice, Academic Press, (1986) pp. 59-1031). Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol. (1984), 133:3001; Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies. Preferably, the binding specificity (i.e., specific immunoreactivity) of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding specificity of the monoclonal antibody can, for example, be determined by the Scatchard analysis of Munson and Pollard (1980), Anal. Biochem., 107:220.

The monoclonal antibodies can also be made by recombinant DNA methods, such as those described in U.S. Pat. No. 4,816,567, which is hereby incorporated by reference in its entirety.

Polyclonal antibodies of the invention can also be produced by various procedures well known in the art.

Antibodies encompassed by the present invention can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds to the antigen of interest can be selected or identified with antigen, e.g., using labeled antigen or antigen bound or captured onto a solid surface or bead. Phage used in these methods are typically filamentous phage including fd and M13 binding domains expressed from phage with Fab, Fv, or disulfide stabilized antibody domains recombinantly fused to either the phage polynucleotide III or polynucleotide VIII protein. Examples of phage display methods that can be used to make the antibodies of the present invention include those disclosed in Brinkman et al. (1995) J. Immunol. Methods, 182:41-50; Ames et al. (1995) J. Immunol. Methods, 184:177-186; Kettleborough et al. (1994) Eur. J. Immunol., 24:952-958; Persic et al. (1997) Gene, 187:9-18; Burton et al. (1994) Advances in Immunology, 57:191-280; PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108, each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below.

Examples of techniques that can be used to produce antibody fragments such as single-chain Fvs and antibodies include those described in U.S. Pat. Nos. 4,946,778 and 5,258,498; Huston et al. (1991) Methods in Enzymology, 203:46-88; Shu et al. (1993) Proc. Natl. Acad. Sci. USA, 90:7995-7999; and Skerra et al. (1988) Science, 240:1038-1040, each of which is incorporated herein by reference in its entirety.

For some uses, including the in vivo use of antibodies in humans and in in vitro detection assays, it may be preferable to use chimeric, humanized, or human antibodies. A chimeric antibody is a molecule in which different portions of the antibody are derived from different animal species, such as antibodies having a variable region derived from a murine monoclonal immunoglobulin and a human immunoglobulin constant region. Methods for producing chimeric antibodies are known in the art. (See, e.g., Morrison (1985), Science, 229:1202; Oi et al. (1986), BioTechniques, 4:214; Gillies et al. (1989), J. Immunol. Methods, 125:191-202; and U.S. Pat. Nos. 5,807,715; 4,816,567; and 4,816,397, which are incorporated herein by reference in their entirety).

Humanized antibodies are antibody molecules from non-human species that bind to the desired antigen and have one or more complementarity determining regions (CDRs) from the nonhuman species and framework regions from a human immunoglobulin molecule. Often, framework residues in the human framework regions are substituted with corresponding residues from the CDR and framework regions of the donor antibody to alter, preferably improve, antigen binding. These framework substitutions are identified by methods well known in the art, e.g., by modeling of the interactions of the CDR and framework residues to identify framework residues important for antigen binding, and by sequence comparison to identify unusual framework residues at particular positions. (See, e.g., Queen et al., U.S. Pat. Nos. 5,693,762 and 5,585,089; and Riechmann et al. (1988) Nature, 332:323, which are incorporated herein by reference in their entireties). Antibodies can be humanized using a variety of techniques known in the art, including, for example, CDR-grafting (EP 239, 400; PCT publication WO 91/09967; U.S. Pat. Nos. 5,225,539; 5,530,101; and 5,585,089); veneering or resurfacing (EP 592,106; EP 519,596; Padlan (1991), Molecular Immunology, 28(4/5):489-498; Studnicka et al. (1994) Protein Engineering, 7(6):805-814; Roguska et al. (1994) Proc. Natl. Acad. Sci. USA, 91:969-973; and chain shuffling (U.S. Pat. No. 5,565,332).

Completely human antibodies are particularly desirable for therapeutic treatment of human patients, so as to avoid or alleviate immune reaction to foreign protein. Human antibodies can be made by a variety of methods known in the art, including the phage display methods described above, using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, WO 91/10741; Lonberg and Huszar (1995) Intl. Rev. Immunol., 13:65-93, WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; 5,939,598; 6,075,181; and 6,114,598, which are incorporated by reference herein in their entirety. In addition, companies such as Abgenix, Inc. (Fremont, Calif.), Protein Design Labs, Inc. (Mountain View, Calif.) and Genpharm (San Jose, Calif.) can be engaged to provide human antibodies directed against a selected antigen using technology similar to the above described technologies.

Once an antibody molecule of the invention has been produced by an animal, a cell line, chemically synthesized, or recombinantly expressed, it can be purified (i.e., isolated) by any method known in the art for the purification of an immunoglobulin or polypeptide molecule, for example, by chromatography (e.g., ion exchange, affinity, particularly by affinity for the specific antigen, Protein A, and sizing column chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

A number of antibody polypeptides that bind specifically to components of the complement cascade have been described and may be used according to the present invention. Antibody polypeptides have been developed which bind to MBL, factor D, factor B, C5, C5a, and C5-9 (see, e.g., Mollnes and Kirschfink, supra, and the references cited therein). A number of antibodies against components of the complement cascade are available from commercial sources such as RDI Research Diagnostics, Inc. (Concord, Mass.). Examples of antibody polypeptides which may be used according to the invention are those on deposit with the ATCC: HB-8327, CRL-1969, HB-8328, and HB-8592.

Analgesia Assays: In Vivo Testing of Agents for Pain Treatment

The invention relates to methods for treatment of pain in an animal. Accordingly, the following section describes assays which can be used to measure or detect pain in an animal, and that can be used to evaluate the effectiveness of a given agent in treating pain via decreasing the activation of the complement cascade. The following assays can also be used to screen candidate compounds, shown to decrease the activation of the complement cascade, for their ability to treat pain in an animal.

Acute Pain

Acute thermal pain is measured on a hot plate mainly in rats or a radiant heat source (Plantar Test, Ugo Basile, Comerio, Italy, Paw thermal stimulator, G. Ozaki, University of California, USA). Two variants of hot plate testing are used: In the classical variant animals are put on a hot surface (52 to 56° C.) and the latency time is measured until the animals show nocifensive behavior, such as stepping or foot licking. The other variant is an increasing temperature hot plate where the experimental animals are put on a surface of neutral temperature. Subsequently this surface is slowly but constantly heated until the animals begin to lick a hind paw. The temperature which is reached when hind paw licking begins is a measure for pain threshold.

Compounds are tested against a vehicle treated control group. Substance application is performed at different time points via different application routes (intravenous (i.v.), intra-peritoneal (i.p.), by mouth (p.o.), by inhalation (i.t.), Intracerebroventricular (i.c.v.), intrathecal, intraspinal, subcutaneous (s.c.), intradermal, or transdermal) prior to pain testing.

According to the invention, a candidate compound, may be administered to an animal which is subjected to an acute pain assay. Acute pain, measured according to the above assay, decreased by at least 10%, and preferably 20%, 40%, 60%, and up to 100% is then indicative of a candidate compound that decreases pain.

Persistent Pain

Persistent pain is measured with the intraplantar formalin or capsaicin test, mainly in rats. A solution of 1 to 5% formalin or 10 to 100 μg capsaicin is injected into one hind paw of the experimental animal. After formalin or capsaicin application the animals show nocifensive reactions like flinching, licking and biting of the affected paw. The number of nocifensive reactions within a time frame of up to 90 minutes is a measure for intensity of pain.

Compounds are tested against a vehicle treated control group. Substance application is performed at different time points via different application routes (i.v., i.p., p.o., i.t., i.c.v., s.c., intrathecal, intraspinal, intradermal, transdermal) prior to formalin or capsaicin administration.

According to the invention, a candidate compound, may be administered to an animal which is subjected to an persistent pain assay. Persistent pain, measured according to the above assay, decreased by at least 10% and preferably 20%, 40%, 60%, and up to 100% is then indicative of a candidate compound that decreases pain.

Neuropathic Pain

Neuropathic pain is induced by different variants of unilateral sciatic nerve injury mainly in rats. The operation is performed under anesthesia. The first variant of sciatic nerve injury is produced by placing loosely constrictive ligatures around the common sciatic nerve (Bennett and Xie, Pain 33 (1988): 87-107). The second variant is the tight ligation of about the half of the diameter of the common sciatic nerve (Seltzer et al., Pain 43 (1990): 205-218). In the next variant, a group of models is used in which tight ligations or transections are made of either the L5 and L6 spinal nerves, or the L5 spinal nerve only (Kim SH; Chung Jm, An experimental-model for peripheral neuropathy produced by segmental spinal nerve ligation in the rat, Pain 50 (3) (1992): 355-363). The fourth variant, the spared nerve injury, involves an axotomy of two of the three terminal branches of the sciatic nerve (tibial and common peroneal nerves) leaving the remaining sural nerve intact whereas the last variant comprises the axotomy of only the tibial branch leaving the sural and common nerves uninjured. Control animals are treated with a sham operation.

Postoperatively, the nerve injured animals develop a chronic mechanical allodynia, cold allodynioa, as well as a thermal hyperalgesia. Mechanical allodynia is measured by means of a pressure transducer (electronic von Frey Anesthesiometer, IITC Inc.-Life Science Instruments, Woodland Hills, SA, USA; Electronic von Frey System, Somedic Sales AB, Hörby, Sweden) or monofilamant von Frey hairs. Thermal hyperalgesia is measured by means of a radiant heat source (Plantar Test, Ugo Basile, Comerio, Italy), hot plate, or by means of a cold plate of 15 to −10° C. where the nocifensive reactions of the affected hind paw are counted as a measure of pain intensity. A further test for cold induced pain is the counting of nocifensive reactions, or duration of nocifensive responses after plantar administration of acetone to the affected hind limb. Chronic pain in general is assessed by registering the circadian rhythms in activity (Surjo and Arndt, Universität zu Köln, Cologne, Germany), and by scoring differences in gait (foot print patterns; FOOTPRINTS program, Klapdor et al., 1997. A low cost method to analyze footprint patterns. J. Neurosci. Methods 75, 49-54). Place preference behavior can also be used.

Compounds are tested against sham operated and vehicle treated control groups. Substance application is performed at different time points via different application routes (i.v., i.p., p.o., i.t., i.c.v., s.c., intrathecal, intraspinal intradermal, transdermal) prior to pain testing.

According to the invention, a candidate compound, may be administered to an animal, which is subjected to an neuropathic pain assay. Neuropathic pain, measured according to the above assay, decreased by at least 10% and preferably 20%, 40%, 60%, and up to 100% is then indicative of a candidate compound that treats pain.

Inflammatory Pain

Inflammatory pain is induced mainly in rats by injection of 0.75 mg carrageenan or 100 μl complete Freund's adjuvant into one hind paw. The animals develop an edema with mechanical allodynia as well as thermal hyperalgesia. Mechanical allodynia is measured by means of a pressure transducer (electronic von Frey Anesthesiometer, IITC Inc.-Life Science Instruments, Woodland Hills, SA, USA) or monofilament von Frey hairs. Thermal hyperalgesia is measured by means of a radiant heat source (Plantar Test, Ugo Basile, Comerio, Italy, Paw thermal stimulator, G. Ozaki, University of California, USA). For edema measurement three methods are being used. In the first method, the animals are sacrificed and the affected hindpaws sectioned and weighed. The second method comprises differences in paw volume by measuring water displacement in a plethysmometer (Ugo Basile, Comerio, Italy). The third method involves measuring paw diameter with a calibrated caliper.

Compounds are tested against uninflamed as well as vehicle treated control groups. Substance application is performed at different time points via different application routes (i.v., i.p., p.o., i.t., i.c.v., s.c., intrathecal, intraspinal, intradermal, transdermal) prior to pain testing.

According to the invention, a candidate compound, may be administered to an animal which is subjected to an inflammatory pain assay. Inflammatory pain, measured according to the above assay, decreased by at least 10% and preferably 20%, 40%, 60%, and up to 100% is then indicative of a candidate compound that treats pain.

Diabetic Neuropathic Pain

Rats treated with a single intraperitoneal injection of 50 to 80 mg/kg streptozotocin develop a profound hyperglycemia and mechanical allodynia within 1 to 3 weeks. Mechanical allodynia is measured by means of a pressure transducer (electronic von Frey Anesthesiometer, IITC Inc.-Life Science Instruments, Woodland Hills, SA, USA) or monofilament von Frey hairs.

Compounds are tested against diabetic and non-diabetic vehicle treated control groups. Substance application is performed at different time points via different application routes (i.v., i.p., p.o., i.t., i.c.v., s.c., intrathecal, intraspinal, intradermal, transdermal) prior to pain testing.

According to the invention, a candidate compound, may be administered to an animal which is subjected to an Diabetic Neuropathic pain assay. Diabetic Neuropathic pain, measured according to the above assay, decreased by at least 10% and preferably 20%, 40%, 60%, and up to 100% is then indicative of a candidate compound that treats pain.

Human Pain

In addition to the pain assays described above, the present invention contemplates that agents that decrease the activity of the complement cascade may be administered to a human to determine if the compound is effective in modulating pain. The level of pain in a human, and thus the effectiveness of a therapeutic compound of the invention may be determined, for example, by a physician, using any clinically relevant scoring method known to those of skill in the art. For example, mechanical pain may be assessed using a Pain Test Algometer (Wagner Instruments, Greenwich, Conn.), monofilament von Frey hairs, thermal pain by peltier or laser devices and pain may be scored in a human using known tests such as the visual analog scale which uses a 100 mm horizontal line marked with “no pain” on one end and “uncontrollable pain” on the other end, or a four-point verbal description of no pain, mild, moderate, or severe pain. Other methods useful for determining the efficacy of pain treatment according to the invention include the peak B endorphin measurement assay (Neuroscience Toolworks, Inc., Evanston, Ill.), the human pain assays described by Fillingim et al. (2004, Anesthesiology 100:1263-1270) functional magnetic resonance imaging (fMRI), the brief pain inventory (BPI), and the McGill questionnaire. Other pain scales and tests may be used according to the general knowledge of those of skill in the art.

Dosage and Administration

Agents of the invention are administered to an animal, preferably in a biologically compatible solution or a pharmaceutically acceptable delivery vehicle, by ingestion, injection, inhalation or any number of other methods. For embodiments where the therapeutic agent is a vector comprising an antisense sequence, or a sequence encoding a ribozyme or siRNA molecule, the vectors may be administered as a pharmaceutical formulation, or may be administered using any method known in the art including microinjection, transfection, transduction, and ex vivo delivery. The dosages administered will vary from patient to patient; a therapeutically effective amount will be that amount of a compound, antibody, antisense polynucleotide, or double stranded RNA molecule that is required to reduce the pain or the symptoms thereof in an animal, for example, at least by 10% or more, 20%, 30%, 40%, 50%, 60%, 70%, 80%, 90%, and up to 100% or more, compared to an animal not treated with the same compound, antibody, antisense polynucleotide, or double stranded RNA molecule, or compared to the same animal before the treatment with the compound, antibody, antisense polynucleotide, or double stranded RNA molecule. A therapeutically effective amount of an agent can also include an amount of a compound, antibody, antisense polynucleotide, or double stranded RNA molecule, that enhances or improves the prophylactic or therapeutic effect(s) of another therapy by at least 10% or more, 20% or more, 30%, 40%, 50%, 60%, 70%, 80%, 90%, and up to 100% or more.

A therapeutic agent according to the invention is preferably administered in a single dose. This dosage may be repeated daily, weekly, monthly, yearly, or until the nucleic acid sequence is no longer differentially expressed.

For animals (patients) suffering from chronic disease requiring long-term therapy, oral, nasal, or rectal application of an agent is preferred to intravenous injection. Alternatively, acute, severe disorders are preferentially treated by intravenous administration of an agent as described herein. Epidural or intrathecal delivery is used to deliver drugs that do not cross the blood brain barrier or have systemic side effects, directly to the spinal cord and dorsal root ganglia. Chronic cannulation of the epidural or subarachnoid space can be used for continuous delivery.

A non-limiting range for a therapeutically or prophylactically effective amount of an agent (e.g., an antibody) useful in the invention is 0.01-20 mg/kg, more preferably 1-10 mg/kg. It is to be noted that dosage values can vary with the type and severity of the pain to be alleviated. It is to be further understood that for any particular subject, specific dosage regimens should be adjusted over time according to the individual need and the professional judgment of the administering clinician.

Where the agent to be administered is an antisense RNA or double stranded RNA molecule, a suitable dose will be in the range of 0.01 to 5.0 milligrams per kilogram body weight of the recipient per day, preferably in the range of 0.1 to 200 micrograms per kilogram body weight per day, more preferably in the range of 0.1 to 100 micrograms per kilogram body weight per day, even more preferably in the range of 1.0 to 50 micrograms per kilogram body weight per day, and most preferably in the range of 1.0 to 25 micrograms per kilogram body weight per day. The pharmaceutical composition may be administered once daily, or may be administered as two, three, four, five, six or more sub-doses at appropriate intervals throughout the day. The dosage unit can also be compounded for delivery over several days, e.g., using a conventional sustained release formulation which provides sustained release of the antisense or dsRNA over a several day period. Sustained release formulations are well known in the art. In this embodiment, the dosage unit contains a corresponding multiple of the daily dose.

Pharmaceutical Compositions

The invention provides for compositions comprising an agent according to the invention admixed with a physiologically compatible carrier. As used herein, “physiologically compatible carrier” refers to a physiologically acceptable diluent such as water, phosphate buffered saline, or saline, and further may include an adjuvant. Adjuvants such as incomplete Freund's adjuvant, aluminum phosphate, aluminum hydroxide, or alum are materials well known in the art.

The invention also provides for pharmaceutical compositions. In addition to the active ingredients, these pharmaceutical compositions may contain suitable pharmaceutically acceptable carrier preparations which is used pharmaceutically.

Pharmaceutical compositions for oral administration are formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use are obtained through a combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl cellulose, hydroxypropylmethyl-cellulose, or sodium carboxymethyl cellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which are used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the invention may be formulated in aqueous solutions, preferably in physiologically compatible buffers such as Hank's solution, Ringer' solution, or physiologically buffered saline. Aqueous injection suspensions may contain substances which increase the viscosity of the suspension, such as sodium carboxymethyl cellulose, sorbitol, or dextran. Additionally, suspensions of the active solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For nasal administration, penetrants appropriate to the particular barrier to be permeated are used in the formulation. Such penetrants are generally known in the art.

The pharmaceutical compositions of the present invention may be manufactured in a manner known in the art, e.g. by means of conventional mixing, dissolving, granulating, dragee-making, levitating, emulsifying, encapsulating, entrapping or lyophilizing processes.

The pharmaceutical composition may be provided as a salt and are formed with many acids, including but not limited to hydrochloric, sulfuric, acetic, lactic, tartaric, malic, succinic, etc. . . . Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5 that is combined with buffer prior to use.

After pharmaceutical compositions comprising a therapeutic agent of the invention formulated in a acceptable carrier have been prepared, they are placed in an appropriate container and labeled for treatment of an indicated condition with information including amount, frequency and method of administration.

### EXAMPLE

Oligonucleotide microarrays were used to measure changes in gene expression in the dorsal horn (DH) and dorsal root ganglia (DRG) of the lumbar spinal cord of rats over time after the SNI, CCI, and SNL nerve injuries to establish both the unique and the shared features of the responses of the peripheral and central nervous systems to mechanical injuries of the peripheral nerve.

Briefly, for SNI, the tibial and common peroneal branches of the sciatic nerve were tightly ligated with a silk suture and transected distally, while the sural nerve was left intact. For CCI, three chromic gut sutures were loosely placed around the sciatic nerve at mid-thigh level. For SNL, an incision was made over the L4/L5 lumbar vertebral column, the transverse processes removed on one side, and a spinal nerve (L4 or L5) tightly ligated. The three models, SNI, CCI, and SNL, all produce a similar pattern of mechanical allodynia and hyperalgesia.

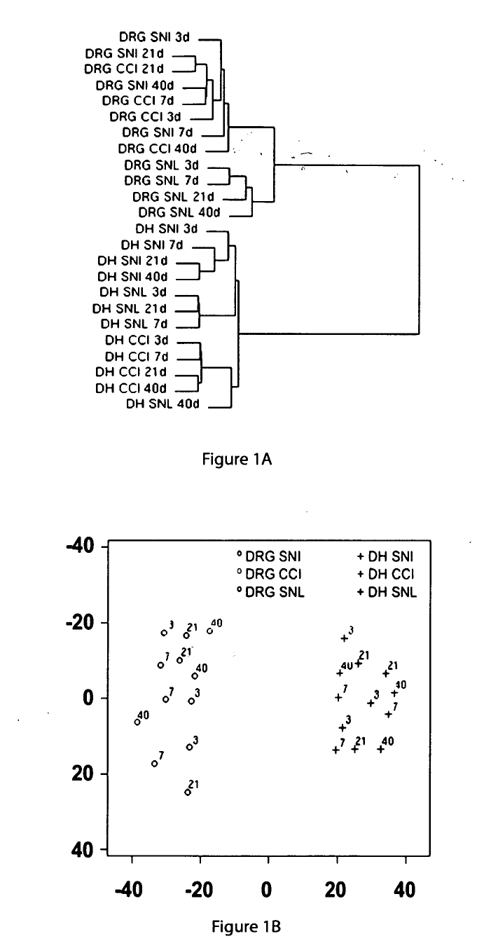

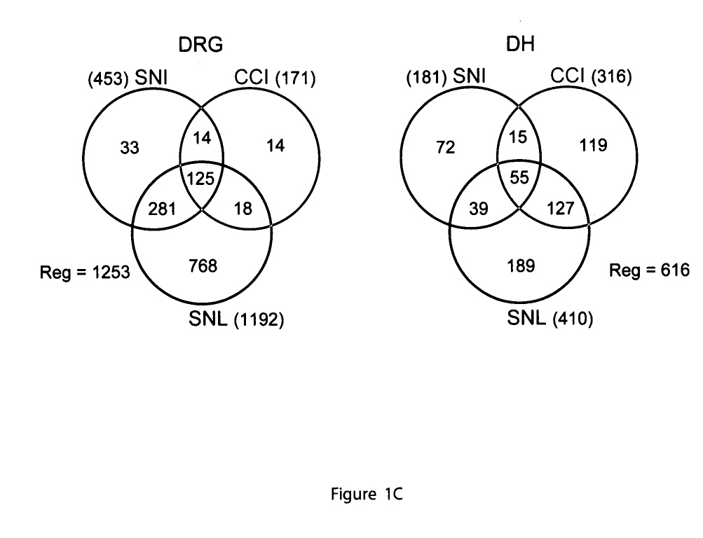

The full data set of 8740 probe sets in the Affymetrix RGU34A array was used to compare global expression profiles for each experimental condition. This analysis had two possible a priori outcomes: either clustering according to time, in which, for example, all the dorsal horn 3 day time points for all nerve injury models would be similar, or alternatively, clustering according to neuropathic pain model, with the four time points for each model clustered separately from those of the other two models. Hierarchical cluster analysis (FIG. 1A) demonstrated that the expression profiles of the DRG were distinct in all cases from the expression profiles of the dorsal horn. Within the dorsal horn, the data clustered according to the nerve injury model. All the time points for the SNI, CCI, and SNL models grouped separately from one another, except for the 40d SNL time point. For the DRG, the SNL model formed a clearly separate cluster (FIG. 1A), but the SNI and CCI models were not separated from each other. Time is a less important contributor to the degree of similarity of the overall data sets than either tissue or the type of injury or tissue. Multidimensional scaling was also used to assess the relationships between the models, displaying the distance matrix in two-dimensional space. The dorsal horn data formed three distinct groups, one for each model (FIG. 1B). The early time points tended to be more similar to one another than the later time points. In the dorsal root ganglion, the CCI and SNI groups were intermingled, with SNL forming a relatively widely dispersed, but separate, region.

The number of genes regulated (defined by p<0.01 and overall fold vs. naive>1.25) for each possible combination of models within the DRG and DH are shown (FIG. 1C). Numbers reported are corrected such that multiple probe sets corresponding to the same UniGene cluster appear only once. Many more genes were regulated in the DRG in the SNL model (1192), which involves transection effectively of all axons of the DRG neurons, as compared to either the SNI (453) or CCI (171) models, where <50% of the neurons are injured. In the dorsal horn, the difference between the models in terms of total number of regulated genes was much less, with fewer genes regulated in the SNI model (181) than in the CCI (316) or SNL (410) models. There was a strong overlap between the SNI and CCI models in the DRG, and between the CCI and SNL models in the dorsal horn (FIG. 1C).

To examine the temporal pattern of gene expression, the distribution of the time to half-peak expression for all genes regulated in each model was measured. In the DRG there was a faster global response to the SNI and SNL than the CCI injury, in spite of the overlap between the SNI and CCI regulated genes in the DRG. In the dorsal horn, the temporal profiles of the three models could not be distinguished from one another. Gene regulation in the dorsal horn for the SNI and SNL models lagged behind that in the DRG, whereas in the CCI model, gene regulation in the dorsal horn occurred faster than in the DRG.

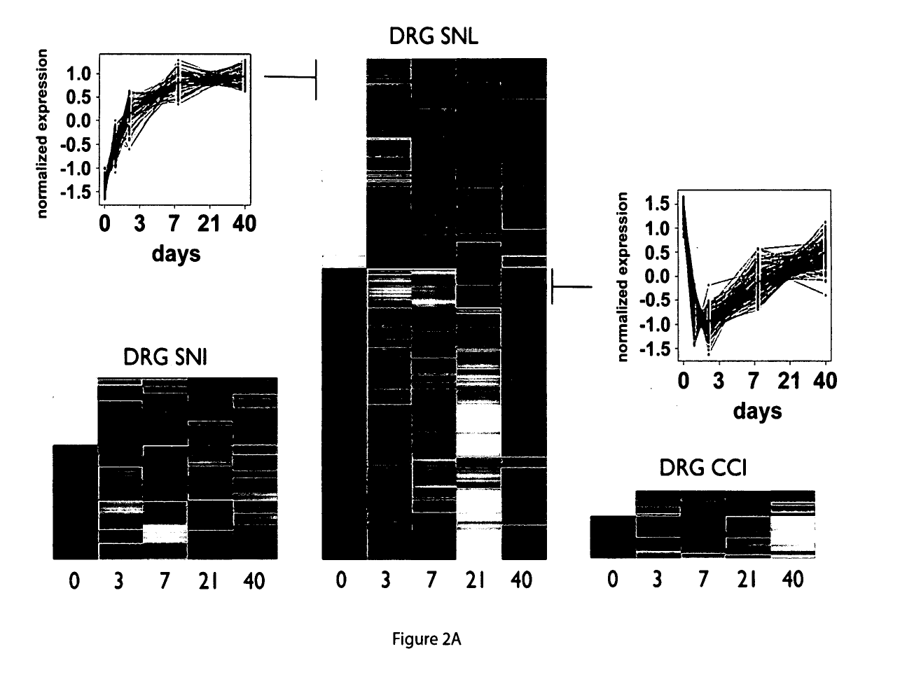

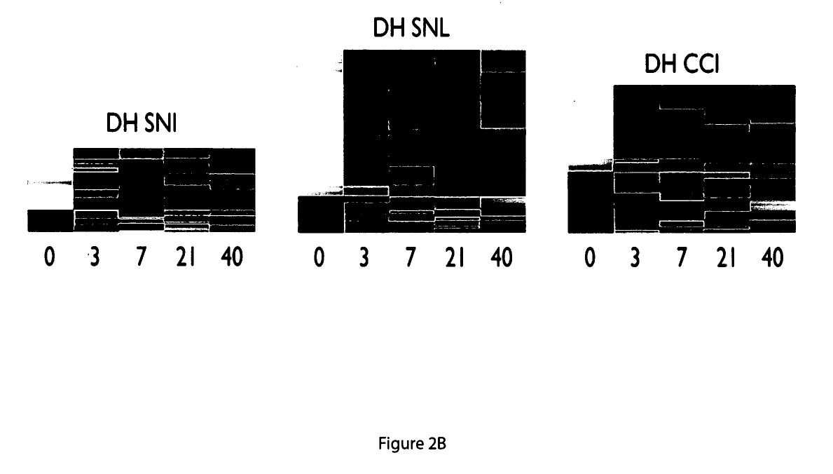

To group the genes according to their change in expression over time within each model two-step clustering was used (Diaz et al., 2002). FIG. 2 illustrates the relative expression of all the regulated genes for all models in the DRG and dorsal horn over the full time course of the experiment. Typically, changes either peaked at 3d with rapid recession to near-naïve values, or showed a relatively sustained pattern of regulation over the full 3d to 40d time course. More genes were down-regulated than up-regulated in the DRG, and vice versa for the DH. The array data in the DRG correlated closely with previously published studies on transcripts whose levels have been documented to change after nerve injury (Costigan et al (2002) BMC Neurosci 3: 16). Further, the extent of regulation in the DRG of seven neuronally expressed genes, measured by in situ hybridization in all three models, was highly consistent with the regulation that was detected by the microarrays.

Those genes that were regulated in all three neuropathic pain models in either the DRG or DH were grouped according to their functional class. This analysis revealed that a substantial proportion of the genes common to all models were associated with immune functions. Other genes regulated in all three models encoded proteins involved in neurotransmission, signaling, transcriptional regulation, metabolism, and the cytoskeleton.

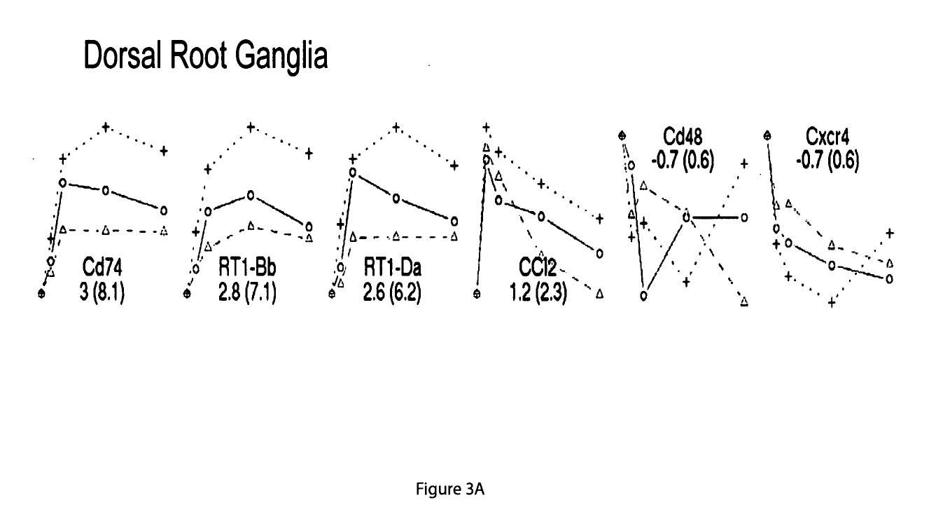

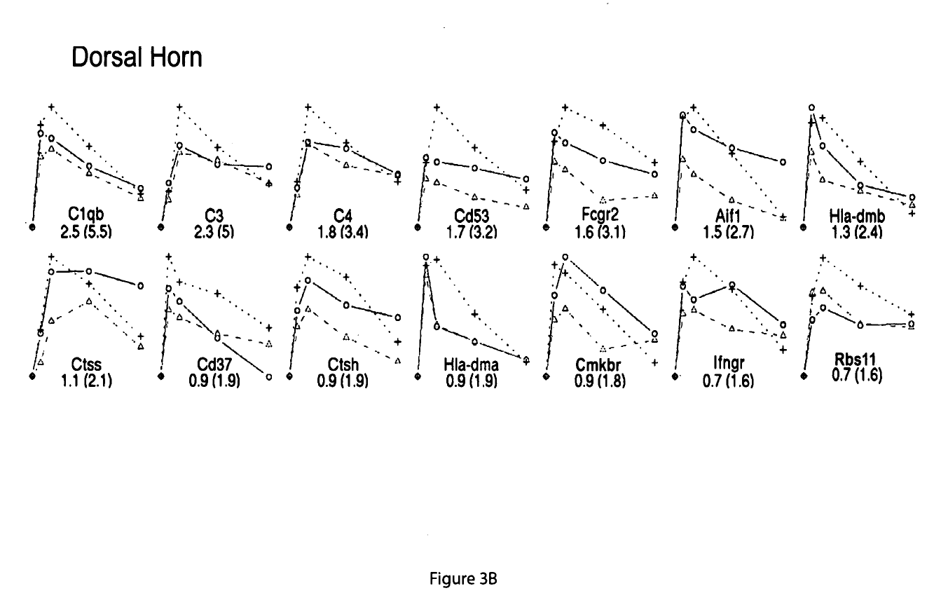

Among the immune genes upregulated in all three models in the DRG (FIG. 3) were MHC class II, the MHC class II associated invariant chain (CD74), and monocyte chemoattractant protein 1 (CCL2). In the dorsal horn, the complement components C1q, C3, and C4, as well as the microglial marker iba1 (aif1), HLA-DMA, HLA-DMB, cathepsin S, cathepsin H, CD37, CD53, the chemokine receptors Rbs11 and Cmkbr5, and the interferon gamma receptor were upregulated in all three models (FIG. 3).

C1, C3, and C4 are components of the complement cascade, an activation and effector mechanism involved in both innate and adaptive immune responses. HLA-DMA, HLA-DMB, and cathepsin S are involved in formation of MHC Class II-peptide complexes by antigen presenting cells (Honey K, Rudensky AY (2003) Nat Rev Immunol. 3: 472-482). These genes are probably markers of macrophage infiltration and microglial activation. CD37 and CD53 are members of the tetraspanin family of membrane proteins; CD37 is expressed primarily in B lymphocytes, while CD53 is expressed in myeloid cells and lymphocytes (Maecker et al., 1997, FASEB J 11: 428-442).

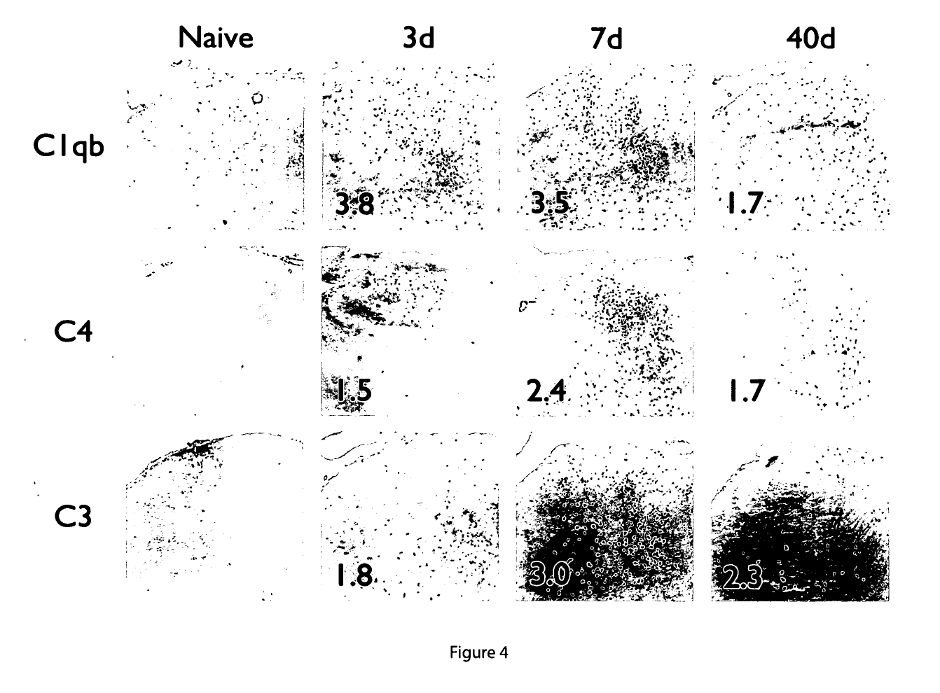

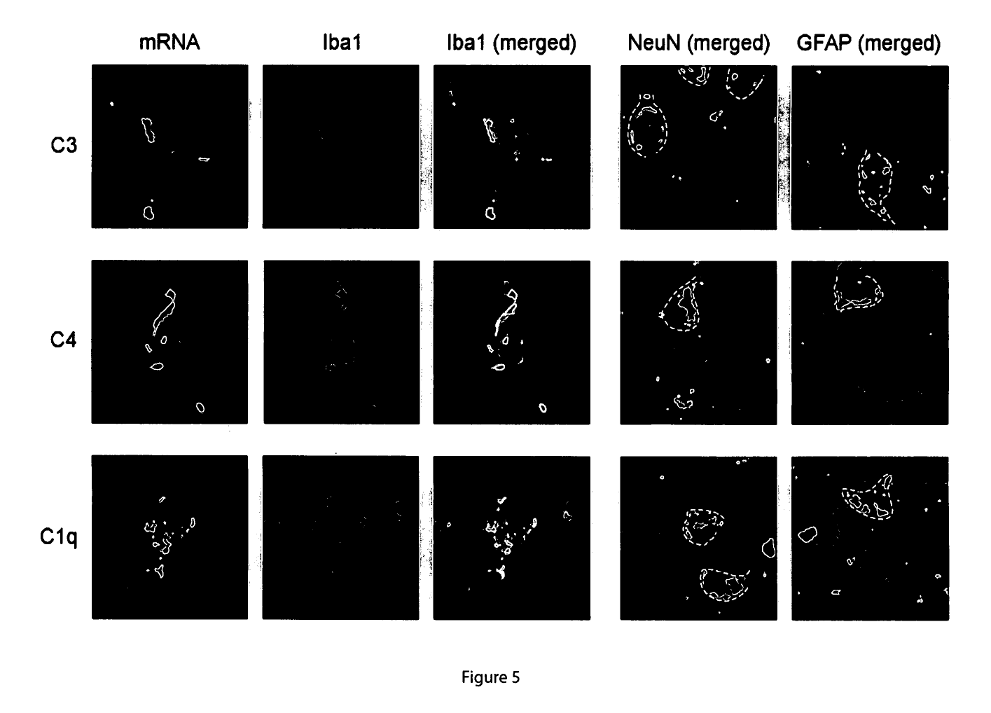

The three most highly regulated genes in the dorsal horn common to all the nerve lesions; C1q, C3, and C4 were characterized by in situ hybridization (FIG. 4). The mRNA expression pattern in the DH for these complement genes shows a temporal regulation that closely matches the array data. In the SNI model, C1q was up-regulated early, peaking at 3d. C3 and C4, were most strongly expressed at 7d after injury. These genes are upregulated in the DH of SNL and CCI animals at e 7d (data not shown). All three of the complement genes were expressed only in myeloid cells, as identified by co-localization with IBA (Imai, 1996) but not in NeuN expressing neurons or in GFAP expressing astrocytes (FIG. 5). Microglia are activated in the DH after nerve injury within the central termination zone of the injured afferents (Tsuda et al., 2005; Winkelstein et al., 2001; Liu et al., 1998).

The expression of C1q, C3, and C4 mRNA in the DRG was also characterized; these genes met the threshold for regulation by microarray in both the SNI and SNL models in the DRG, but not in the CCI model. The in situ data indicated that C1q, C4, and C3 were all up-regulated in non-neuronal cells within the DRG. C1q and C4 were more markedly upregulated than C3.

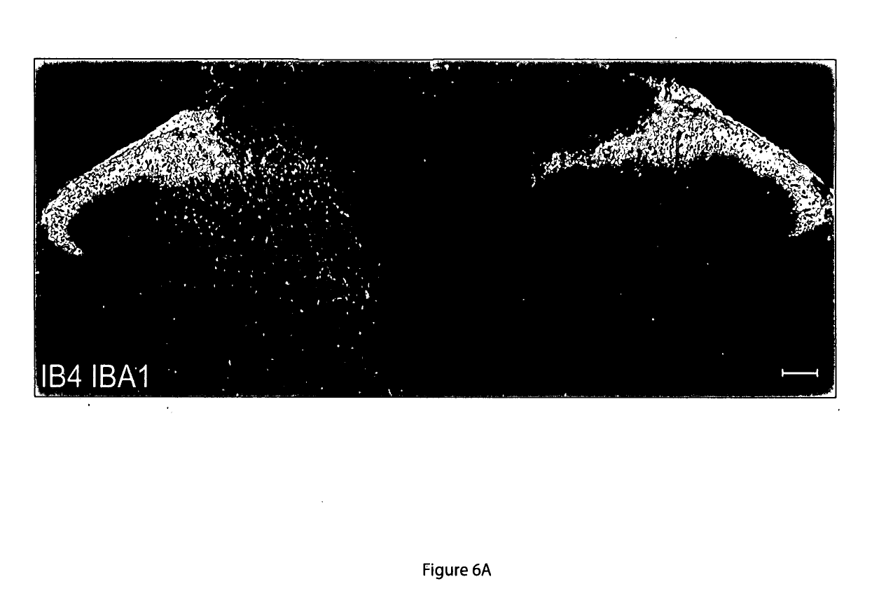

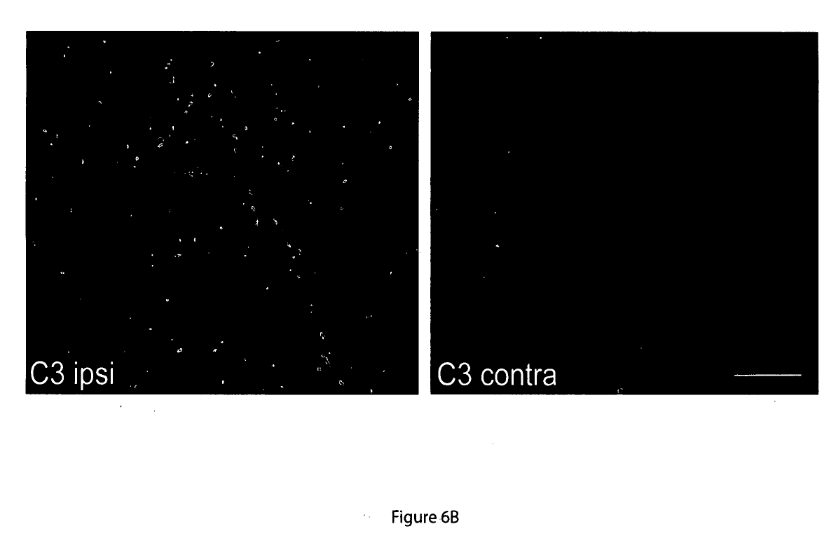

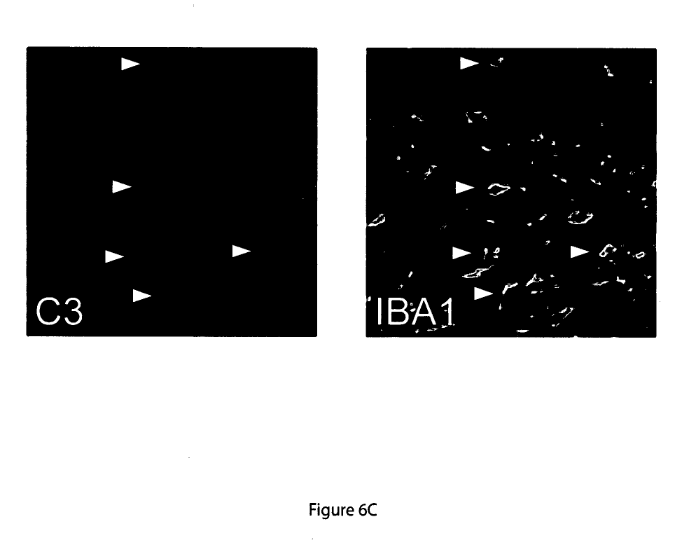

In order to define the cellular and topographical localization of complement C3 protein in the dorsal horn following nerve injury C3, IBA and a marker of C-fiber nociceptor central terminals (IB4) after SNI were stained for (FIG. 6). It was found that C3 immunoreactivity co-localized to a subset of the IBA positive cells (microglia/macrophages) in the DH ipsilateral to the injury. Within lamina II, C3 immunoreactivity was strongest in, but not limited to, the area innervated by injured nociceptive afferents, as detected by a reduction in IB4 staining. IB4 staining decreases after peripheral nerve injury in the central terminal zone of injured afferents (Munglani et al., 1995; Shehab et al., 2004).

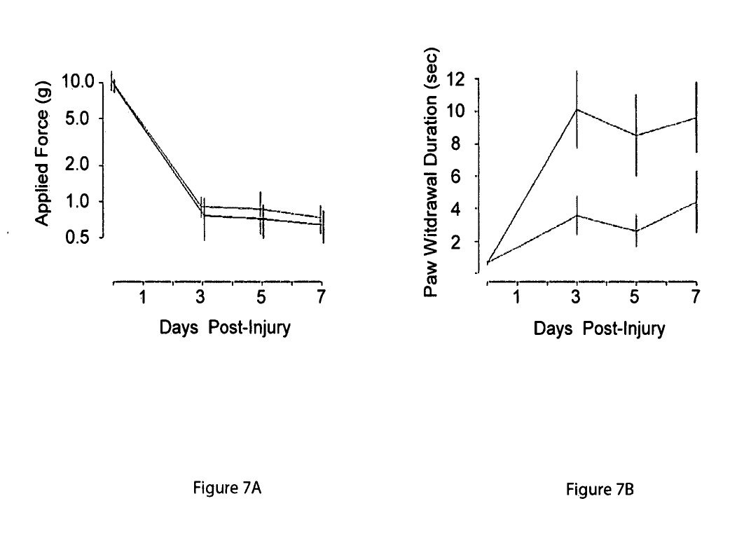

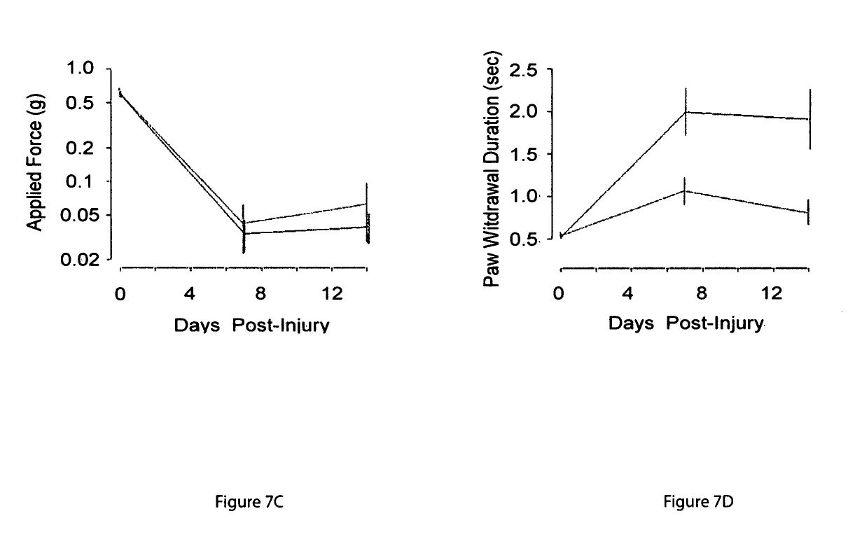

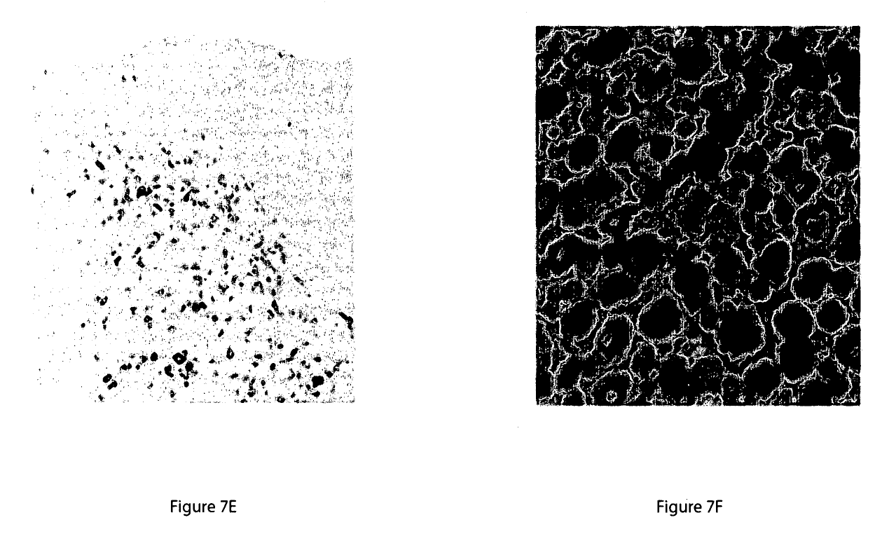

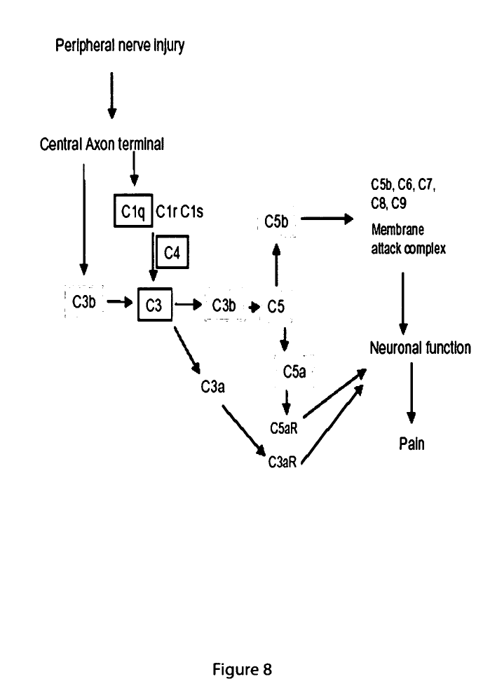

To test whether spinal cord complement is necessary for the behavioral manifestations of neuropathic pain, complement in the spinal cord of SNI rats was depleted by administering cobra venom factor (CVF) intrathecally. CVF has activity similar to activated C3, and rapidly depletes or consumes all available complement. No difference in either the mechanical von Frey threshold or the pinprick test was observed in uninjured naïve animals treated intrathecally with CVF. After SNI however, a significant reduction in mechanical hyperalgesia (pinprick response; FIG. 7B) but not mechanical allodynia (von Frey threshold; FIG. 7A) was found in rats treated with CVF. CVF applied intraperitoneally at the same dose as that used intrathecally did not produce serum decomplementation or any detectable effect on mechanical sensitivity (data not shown).

It was also tested whether mice deficient in complement component C5 have an impaired behavioral response to nerve injury. C5 is necessary for two of the major effector functions of complement: release of anaphalotoxin peptide C5a and production and formation of the membrane attack complex (MAC). No difference in mechanical threshold to von Frey hairs was observed in C5 deficient, uninjured mice (FIG. 7C), but these mice showed reduced pinprick hyperalgesia after SNI as compared to a congenic control strain (FIG. 7D).

CD59 is a GPI-anchored protein that acts to prevent the formation of the membrane attack complex (MAC) in cells that express it, and is an important mechanism conferring MAC-resistance on most nucleated cell types (Baalsubramanian, 2004, J Immunol 173: 3684-3692). In the DRG, all neurons, but no non-neuronal cells expressed CD59 (FIG. 7E). In the DH, CD59 was expressed in a ventrodorsal gradient, with strong expression in ventral motor neurons, moderate expression in some neurons in the deep dorsal horn, and little or no expression in the superficial laminae (FIG. 7F).

Taken together, these studies teach that it is the C5 dependent actions of complement that contribute to neuropathic pain. It is clear from the foregoing that a major shared response to multiple forms of peripheral nerve injury is immunologic gene activation in myeloid cells and particularly prominent amongst these is induction of complement genes. Furthermore depletion of complement in the spinal cord or testing animals deficient in C5 attenuates pain hypersensitivity. It is concluded therefore that a neuroimmune interaction involving complement underlies in part the maladaptive responses to nerve injury that generate neuropathic pain, and thus decreasing the activation of the complement cascade is a useful method for the treatment of pain.

The foregoing studies utilized the following methodologies:

Animal surgery. Five separate experimental groups were prepared for the microarray experiments: SNI, CCI, SNL, sham SNI/CCI, and sham SNL. Adult male Sprague-Dawley rats were anesthetized using isoflurane and surgery undertaken as described before (Decosterd and Woolf, 2000; Bennett, 1988; Kim and Chung, 1992). All procedures were implemented according to Massachusetts General Hospital animal care regulations.

Tissue preparation, RNA extraction and chip hybridization. Tissue samples were obtained three, seven, twenty-one, and forty days after the lesions/sham surgery. The left L4 and L5 DRGs were rapidly dissected and frozen at −80° C. For the SNL animals, only the DRG whose segmental nerve was injured was used. The ipsilateral lumbar L4 and L5 dorsal horn from the same animals was dissected and frozen. The tissues were then homogenized, and total RNA was obtained by acid phenol extraction (TRIzol reagent, Invitrogen, Carlsbad, Calif.). Biotinylated cRNA for hybridization was produced from the total RNA and hybridized to the Affymetrix RGU34A chip (Costigan et al., 2002). In each experimental condition three biologically independent hybridizations were performed, each using cRNA probes produced from independent RNA samples extracted from pooled tissue from five animals.

Data analysis. CEL files were produced using MAS 5.0. All other data analysis was done using R software (R Development Core Team, 2005). Background correction and quantile-quantile data normalization were performed, followed by calculation of probe set intensities using the RMA (robust multiarray average) method (Irizarry et al., 2003; Bolstad et al., 2003)(www.bioconductor.org; Gentleman et al., 2004). To assess reproducibility, the correlation coefficients between all possible pairings of single chips within a triplicate were calculated. For presentation, the weakest of the three correlation coefficients was used to represent that triplicate. The histogram of these worst correlation coefficients (Supplemental FIG. 5A) demonstrates that the data was highly reproducible: the worst correlation coefficient was still better than 0.97.

The iteratively re-weighted least squares regression method was used to estimate the expression level for each gene in each DRG and DH experiment (Venables and Ripley, 2002; Diaz et al., 2003). The regression model treated time points (3, 7, 21, 40) as nested within the type of nerve injury (SNI, CCI, SNL, sham SNI/CCI, sham SNL). Bootstrap P values associated with contrasts between SNI and Naïve, CCI and Naïve, and SNL and Naïve for each gene were calculated by re-sampling from the residuals of the original model. The threshold P value consistent with a false discovery rate of 5% was identified as 0.01 (Storey and Tibshirani, 2003), based on an estimate of the overall proportion of true null hypotheses derived from the observed distribution of p values

(Supplemental FIG. 5B). The q-value calculation was carried out separately for each injury within each tissue, and an overall p value threshold of 0.01 selected because it resulted in a q value near 5% for each model (DRG SNI 3.7%, DRG CCI 7.2%, DRG SNL 1.5%; DH SNI 5.4%; DH CCI 4.6%; DH SNL 2.3%). In addition to the p value threshold, it was required that the contrast between naïve and nerve injury, essentially the expression ratio relative to naïve averaged over the four post-injury time points, reach at least 1.25 fold, when converted to a linear scale, for a gene to be considered differentially expressed. This allows the inclusion of genes that are modestly differentially expressed, but persist over time, along with genes that are transiently expressed but reach high peak or trough levels. The sham controls were used to assess probe set-specific variability. Many genes show changes in expression to even minimal nerve injury, using the sham controls as explicit filters for eliminating genes would have biased the results.

After estimating the expression level, the data from all the probe sets were used for hierarchical cluster analysis. The Euclidean distance and average linkage were used (Kaufman and Rosseeuw, 1990). In addition, Sammon's nonlinear mapping was implemented on the matrix of Euclidean distances using the MASS R library (Venables and Ripley, 2002).

For assessment of the temporal responses of the regulated genes, two analyses were carried out, one quantitative and one graphical. First, the time required for each gene to reach its half-maximal expression level was calculated. Linear interpolation was used to estimate expression at intermediate times. The empirical cumulative distribution function of half-maximal times for each model within each tissue was calculated. Second, the coordinated temporal behavior of groups of genes was assessed graphically. The data for each probe set were scaled to mean zero, root mean square 1 over the data for a single model in a given tissue (Tavazoie et al, 1998). The scaled data were then grouped using k-means cluster analysis (Hartigan and Wong, 1979). The number of clusters was chosen empirically, by finding the elbow in the plot of the total within cluster sum of squares as a function of cluster number. The means of the k-means clusters were grouped using divisive hierarchical clustering (Kaufman and Rosseeuw, 1990).

In situ hybridization. Tissue was rapidly removed, embedded in Tissue Tek OCT (Sakura, Torrance, Calif.) and frozen. Sections were cut serially at 18 μm, and in situ hybridization histochemistry was performed using digoxygenin-labeled antisense riboprobes (0.6 to 2 kb in length) (Blackshaw and Snyder, 1997).

Immunohistochemistry. Rats were perfused with 0.9% NaCl, followed by 4% paraformaldehyde in 0.025% picric acid, 1X PBS. 12 micron sections were prepared, washed, blocked, then incubated for 24 hours in 1% BSA with 0.1% Triton X-100 in 1×PBS, with 1:1000 goat anti-rat C3 (MP Bio, Irvine, Calif.). Colocalization was carried out using 1:750 rabbit anti-rat IBA (Wako, Richmond, Va.) and 1:100 Griffonia simplicifonica isolectin IB4 conjugated to FITC (Sigma-Aldrich, St. Louis, Mo.).

Animal behavior. Punctate mechanical pain threshold using calibrated monofilament von Frey hairs, and the duration of response to a standard pinprick were tested as described before (Decosterd and Woolf, 2000). The behavioral tests were done on 7 or 8 animals for each of the models, with two baseline pre-surgery time points, and three, seven, twenty-one, and forty days after the nerve injury. Behavioral testing was done with the experimenter blinded to the nerve injury condition. For cobra venom factor (CVF) treatment, 1 unit of CVF (Quidel, San Diego, Calif. ) in 200 μlof 0.9% NaCl was infused using an Alzet osmotic pump (Durect, Cupertino, Calif.) connected to an intrathecal catheteter, at a rate of 1 μL/hour. Pump and catheter placement, with initiation of infusion, was carried out 24 hours prior to SNI surgery. In an additional control experiment, rats were treated with the same total dose divided into daily injections supplied intraperitoneally in 0.5 mL. Behavioral tests were performed on C5 deficient and congenic wildtype mice obtained from The Jackson Laboratory (Bar Harbor, Me.).

### Other Embodiments

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

1. A method of treating or preventing pain in a mammal comprising administering to said mammal an antisense polynucleotide capable of inhibiting the expression of a polynucleotide sequence that encodes a component of the complement cascade.

2. A method of treating or preventing pain in a mammal comprising administering to said mammal a double stranded RNA molecule wherein one of the strands of said double stranded RNA molecule is identical to at least 10 contiguous residues of an mRNA transcript obtained from a polynucleotide sequence encoding a of the component of the complement cascade.

3. A method of treating or preventing pain in a mammal in need thereof, comprising:
administering to said mammal a therapeutically effective amount of an agent which decreases the activity of a components of the complement cascade, wherein said agent is selected from the group consisting of soluble complement receptor type 1, soluble complement receptor type 1 lacking long homologous repeat-A, soluble complement receptor type 1-sialyl lewis, complement receptor type 2, soluble decay accelerating factor, soluble membrane cofactor protein, soluble CD59, decay accelerating factor-CD59 hybrid, membrane cofactor protein-decay accelerating factor hybrid, C1 inhibitor, C1q receptor, C3, C3a, C089, PR226, CBP2, DFP, BCX-1470, TKIXc, K-76 COOH, FUT-175, PS-oligo, Glycyrrhizin, GR-2II, AGIIb-1, AR-2IIa, Rosmarinic acid, BR-5-I, Fucan, complestatin, decorin, dextran, heparin, LU51198, GCRF, CSPG, C4 inactivator, compstatin, CR1 (CD35), CD2 (CD21), MCP (CD46), DAF (CD55), factor H, C3BP, Crry, TP-10, plasma-derived protein C1 esterase inhibitor, vaccinia virus complement control protein, AcF[OPdCHaWR], CGS32359, 3D53, SB-290157, and cobra venom factor.

4. The method of claim 3, wherein said agent decreases the activity of a complement component selected from the group consisting of C3, C3a, C5, and C5a.

5. A method of treating or preventing pain in a mammal in need thereof, comprising:
administering a therapeutically effective amount of an antibody polypeptide which binds to a component of the complement cascade.

6. A pharmaceutical formulation comprising an agent selected from the group consisting of soluble complement receptor type 1, soluble complement receptor type 1 lacking long homologous repeat-A, soluble complement receptor type 1-sialyl lewis, complement receptor type 2, soluble decay accelerating factor, soluble membrane cofactor protein, soluble CD59, decay accelerating factor-CD59 hybrid, membrane cofactor protein-decay accelerating factor hybrid, C1 inhibitor, C1q receptor, C089, PR226, CBP2, DFP, BCX-1470, TKIXc, K-76 COOH, FUT-175, PS-oligo, Glycyrrhizin, GR-2II, AGIIb-1, AR-2IIa, Rosmarinic acid, BR-5-I, Fucan, complestatin, decorin, dextran, heparin, LU51198, GCRF, CSPG, C4 inactivator, compstatin, CR1 (CD35), CD2 (CD21), MCP (CD46), DAF (CD55), factor H, C3BP, Crry, TP-10, plasma-derived protein C1 esterase inhibitor, vaccinia virus complement control protein, AcF[OPdCHaWR], CGS32359, 3D53, SB-290157, and cobra venom factor, and a carrier.

7. A pharmaceutical formulation comprising an antibody polypeptide which binds to a component of the complement cascade, and a carrier.

8. A pharmaceutical formulation comprising an antisense polynucleotide that inhibits the expression of a polynucleotide sequence that encodes a component of the complement cascade, and a carrier.


SEQUENCE LISTING (108 sequences)

SEQ ID NO: 1 - DNA, 22 bp, Homo sapiens
atatctcccaggcccagctcag

SEQ ID NO: 2 - DNA, 22 bp, Homo sapiens
ggcccagctcagctgcaccggg

SEQ ID NO: 3 - DNA, 22 bp, Homo sapiens
agctgcaccgggcccccagcca

SEQ ID NO: 4 - DNA, 22 bp, Homo sapiens
ggcccccagccatccctggcat

SEQ ID NO: 5 - DNA, 22 bp, Homo sapiens
ttctgtgggcaactgggttctc

SEQ ID NO: 6 - DNA, 22 bp, Homo sapiens
tgtgggcaactgggttctccac

SEQ ID NO: 7 - DNA, 22 bp, Homo sapiens
gggcaactgggttctccactgg

SEQ ID NO: 8 - DNA, 22 bp, Homo sapiens
caactgggttctccactgggca

SEQ ID NO: 9 - DNA, 22 bp, Homo sapiens
gcctgctacacggcccactcgg

SEQ ID NO: 10 - DNA, 22 bp, Homo sapiens
tgctacacggcccactcgggca

SEQ ID NO: 11 - DNA, 22 bp, Homo sapiens
tacacggcccactcgggcaagc

SEQ ID NO: 12 - DNA, 22 bp, Homo sapiens
acggcccactcgggcaagctga

SEQ ID NO: 13 - DNA, 22 bp, Homo sapiens
aatatctcgggtggcaccttca

SEQ ID NO: 14 - DNA, 22 bp, Homo sapiens
atctcgggtggcaccttcaccc

SEQ ID NO: 15 - DNA, 22 bp, Homo sapiens
tcgggtggcaccttcaccctca

SEQ ID NO: 16 - DNA, 22 bp, Homo sapiens
ggtggcaccttcaccctcagcc

SEQ ID NO: 17 - DNA, 19 bp, Homo sapiens
tcatctgggggtcccccta

SEQ ID NO: 18 - DNA, 19 bp, Homo sapiens
tctgggggtccccctatcg

SEQ ID NO: 19 - DNA, 19 bp, Homo sapiens
gggggtccccctatcggtg

SEQ ID NO: 20 - DNA, 19 bp, Homo sapiens
ggtccccctatcggtgggg

SEQ ID NO: 21 - DNA, 22 bp, Homo sapiens
ctgagctcgggggccaaggtgc

SEQ ID NO: 22 - DNA, 22 bp, Homo sapiens
agctcgggggccaaggtgctgg

SEQ ID NO: 23 - DNA, 22 bp, Homo sapiens
tcgggggccaaggtgctggcca

SEQ ID NO: 24 - DNA, 22 bp, Homo sapiens
ggggccaaggtgctggccacgc

SEQ ID NO: 25 - DNA, 22 bp, Homo sapiens
ctccaagagggccaggcactgg

SEQ ID NO: 26 - DNA, 22 bp, Homo sapiens
caagagggccaggcactggagt

SEQ ID NO: 27 - DNA, 22 bp, Homo sapiens
gagggccaggcactggagtacg

SEQ ID NO: 28 - DNA, 22 bp, Homo sapiens
ggccaggcactggagtacgtgt

SEQ ID NO: 29 - DNA, 22 bp, Homo sapiens
gcagttctggtcctcctaggag

SEQ ID NO: 30 - DNA, 22 bp, Homo sapiens
gttctggtcctcctaggagcgg

SEQ ID NO: 31 - DNA, 22 bp, Homo sapiens
ctggtcctcctaggagcggccg

SEQ ID NO: 32 - DNA, 22 bp, Homo sapiens
gtcctcctaggagcggccgcct

SEQ ID NO: 33 - DNA, 22 bp, Homo sapiens
gcaaaaaactagtgctgtccag

SEQ ID NO: 34 - DNA, 22 bp, Homo sapiens
aaaaactagtgctgtccagtga

SEQ ID NO: 35 - DNA, 22 bp, Homo sapiens
aactagtgctgtccagtgagaa

SEQ ID NO: 36 - DNA, 22 bp, Homo sapiens
tagtgctgtccagtgagaagac

SEQ ID NO: 37 - DNA, 22 bp, Homo sapiens
actgtggctaagtgtggggacc

SEQ ID NO: 38 - DNA, 22 bp, Homo sapiens
gtggctaagtgtggggaccaga

SEQ ID NO: 39 - DNA, 22 bp, Homo sapiens
gctaagtgtggggaccagacag

SEQ ID NO: 40 - DNA, 22 bp, Homo sapiens
aagtgtggggaccagacaggac

SEQ ID NO: 41 - DNA, 22 bp, Homo sapiens
atttagttactcctcaggccat

SEQ ID NO: 42 - DNA, 22 bp, Homo sapiens
tagttactcctcaggccatgtt

SEQ ID NO: 43 - DNA, 22 bp, Homo sapiens
ttactcctcaggccatgttcat

SEQ ID NO: 44 - DNA, 22 bp, Homo sapiens
ctcctcaggccatgttcattta

SEQ ID NO: 45 - DNA, 22 bp, Homo sapiens
atgaactccttcaattatacca

SEQ ID NO: 46 - DNA, 22 bp, Homo sapiens
aactccttcaattataccaccc

SEQ ID NO: 47 - DNA, 22 bp, Homo sapiens
tccttcaattataccacccctg

SEQ ID NO: 48 - DNA, 22 bp, Homo sapiens
ttcaattataccacccctgatt

SEQ ID NO: 49 - DNA, 22 bp, Homo sapiens
tcaaaaacttgcaattctggaa

SEQ ID NO: 50 - DNA, 22 bp, Homo sapiens
aaaacttgcaattctggaaccc

SEQ ID NO: 51 - DNA, 22 bp, Homo sapiens
acttgcaattctggaacccaga

SEQ ID NO: 52 - DNA, 22 bp, Homo sapiens
tgcaattctggaacccagagca

SEQ ID NO: 53 - DNA, 22 bp, Homo sapiens
atgaaggtgataagcttattca

SEQ ID NO: 54 - DNA, 22 bp, Homo sapiens
aaggtgataagcttattcattt

SEQ ID NO: 55 - DNA, 22 bp, Homo sapiens
gtgataagcttattcattttgg

SEQ ID NO: 56 - DNA, 22 bp, Homo sapiens
ataagcttattcattttggtgg

SEQ ID NO: 57 - DNA, 22 bp, Homo sapiens
ggcactcacagcacaggcttgt

SEQ ID NO: 58 - DNA, 22 bp, Homo sapiens
actcacagcacaggcttgttat

SEQ ID NO: 59 - DNA, 22 bp, Homo sapiens
cacagcacaggcttgttatggg

SEQ ID NO: 60 - DNA, 22 bp, Homo sapiens
agcacaggcttgttatgggtct

SEQ ID NO: 61 - DNA, 22 bp, Homo sapiens
ttttttttttcatcctactttg

SEQ ID NO: 62 - DNA, 22 bp, Homo sapiens
tttttttcatcctactttgttt

SEQ ID NO: 63 - DNA, 22 bp, Homo sapiens
ttttcatcctactttgttttat

SEQ ID NO: 64 - DNA, 22 bp, Homo sapiens
tcatcctactttgttttattgg

SEQ ID NO: 65 - DNA, 22 bp, Homo sapiens
cagcatgtcagcctgccggagc

SEQ ID NO: 66 - DNA, 22 bp, Homo sapiens
catgtcagcctgccggagcttt

SEQ ID NO: 67 - DNA, 22 bp, Homo sapiens
gtcagcctgccggagctttgca

SEQ ID NO: 68 - DNA, 22 bp, Homo sapiens
agcctgccggagctttgcagtt

SEQ ID NO: 69 - DNA, 22 bp, Homo sapiens
ctgatttacaggaactcacacc

SEQ ID NO: 70 - DNA, 22 bp, Homo sapiens
atttacaggaactcacaccagc

SEQ ID NO: 71 - DNA, 22 bp, Homo sapiens
tacaggaactcacaccagcgat

SEQ ID NO: 72 - DNA, 22 bp, Homo sapiens
aggaactcacaccagcgatcaa

SEQ ID NO: 73 - DNA, 22 bp, Homo sapiens
aaaactctgatctggggaggaa

SEQ ID NO: 74 - DNA, 22 bp, Homo sapiens
actctgatctggggaggaacca

SEQ ID NO: 75 - DNA, 22 bp, Homo sapiens
ctgatctggggaggaaccagga

SEQ ID NO: 76 - DNA, 22 bp, Homo sapiens
atctggggaggaaccaggacta

SEQ ID NO: 77 - DNA, 22 bp, Homo sapiens
attctgtctttcacatacattg

SEQ ID NO: 78 - DNA, 22 bp, Homo sapiens
ctgtctttcacatacattgaga

SEQ ID NO: 79 - DNA, 22 bp, Homo sapiens
tctttcacatacattgagacca

SEQ ID NO: 80 - DNA, 22 bp, Homo sapiens
ttcacatacattgagaccaaaa

SEQ ID NO: 81 - DNA, 22 bp, Homo sapiens
ggaattcgggcacgagtgaaag

SEQ ID NO: 82 - DNA, 22 bp, Homo sapiens
attcgggcacgagtgaaagatt

SEQ ID NO: 83 - DNA, 22 bp, Homo sapiens
cgggcacgagtgaaagatttca

SEQ ID NO: 84 - DNA, 22 bp, Homo sapiens
gcacgagtgaaagatttcaaac

SEQ ID NO: 85 - DNA, 22 bp, Homo sapiens
cgaacacctccaacatgaagct

SEQ ID NO: 86 - DNA, 22 bp, Homo sapiens
acacctccaacatgaagcttct

SEQ ID NO: 87 - DNA, 22 bp, Homo sapiens
cctccaacatgaagcttcttca

SEQ ID NO: 88 - DNA, 22 bp, Homo sapiens
ccaacatgaagcttcttcatgt

SEQ ID NO: 89 - DNA, 22 bp, Homo sapiens
caatcatggatcaatagctatg

SEQ ID NO: 90 - DNA, 22 bp, Homo sapiens
tcatggatcaatagctatgttt

SEQ ID NO: 91 - DNA, 22 bp, Homo sapiens
tggatcaatagctatgtttgga

SEQ ID NO: 92 - DNA, 22 bp, Homo sapiens
atcaatagctatgtttggagaa

SEQ ID NO: 93 - DNA, 22 bp, Homo sapiens
acactctgggcgcggagcacaa

SEQ ID NO: 94 - DNA, 22 bp, Homo sapiens
ctctgggcgcggagcacaatga

SEQ ID NO: 95 - DNA, 22 bp, Homo sapiens
tgggcgcggagcacaatgattg

SEQ ID NO: 96 - DNA, 22 bp, Homo sapiens
gcgcggagcacaatgattggtc

SEQ ID NO: 97 - DNA, 22 bp, Homo sapiens
cccggggcgtatgacgccggag

SEQ ID NO: 98 - DNA, 22 bp, Homo sapiens
ggggcgtatgacgccggagccc

SEQ ID NO: 99 - DNA, 22 bp, Homo sapiens
gcgtatgacgccggagccctct

SEQ ID NO: 100 - DNA, 22 bp, Homo sapiens
tatgacgccggagccctctgac

SEQ ID NO: 101 - DNA, 22 bp, Homo sapiens
gggccggggggcggagccttgc

SEQ ID NO: 102 - DNA, 22 bp, Homo sapiens
ccggggggcggagccttgcggg

SEQ ID NO: 103 - DNA, 22 bp, Homo sapiens
gggggcggagccttgcgggctg

SEQ ID NO: 104 - DNA, 22 bp, Homo sapiens
ggcggagccttgcgggctggag

SEQ ID NO: 105 - DNA, 22 bp, Homo sapiens
cttttatctcttaggaaatcaa

SEQ ID NO: 106 - DNA, 22 bp, Homo sapiens
ttatctcttaggaaatcaaaga

SEQ ID NO: 107 - DNA, 22 bp, Homo sapiens
tctcttaggaaatcaaagagca

SEQ ID NO: 108 - DNA, 22 bp, Homo sapiens
cttaggaaatcaaagagcagga